(19) United States
(12) Patent Application Publication
(10) Pub. No.: US 2024/0316307 A1
(43) Pub. Date: Sep. 26, 2024

(54) TONGUE MUSCLE SPINDLE STIMULATOR

(71) Applicant: Harbinger AS, Hønefoss (NO)

(72) Inventors: Gitle KIRKESOLA, Hønefoss (NO); Geir Atle EKAAS, Hønefoss (NO)

(21) Appl. No.: 18/614,022

(22) Filed: Mar. 22, 2024

Related U.S. Application Data
(60) Provisional application No. 63/492,104, filed on Mar. 24, 2023.

(51) Int. Cl. A61M 21/00; A61H 23/02

(52) U.S. Cl. CPC A61M 21/00; A61H 23/0236; A61H 2201/0157; A61H 2201/5005; A61M 2021/0022; A61M 2210/0643

(57) ABSTRACT

A device and method for stimulating tongue muscle spindles via stochastic resonance is disclosed. The device can include a stimulator for application to the tongue of a user. The stimulator can include a transducer configured to produce vibrations, a tongue pad coupled to the transducer, an amplifier, and a controller. The method can include selecting a type of vibrations to be at least one of Brownian, pink, or white noise, selecting a frequency range of the vibrations, and applying a stimulator to the tongue of a user to apply the vibrations to the tongue of the user.

### CROSS-REFERENCE TO RELATED APPLICATIONS

This application claims the benefit of U.S. Provisional Application No. 63/492,104, filed Mar. 24, 2023, the disclosure of which is incorporated herein by reference in its entirety.

### BACKGROUND

Often, individuals can suffer from a wide variety of symptoms, such as anxiety, cognitive problems, chronic pain, etc. Cognitive problems can include loss of memory, difficulty finding the right words, trouble focusing, etc. Pain can be intermittent or long-lasting. Symptoms like these can greatly affect the individual's ability to function normally in daily life, tolerate work, exercise, etc. Chronic pain can have a negative effect on brain function. Additionally, treatment of such symptoms can require significant expense of time and money and can interfere with an individual's daily activities.

### BRIEF SUMMARY

Accordingly, there is a need to provide a treatment device which is portable, easily accessible to an individual, and inexpensive compared to other medical treatments or medications.

Aspects of a device according to the present disclosure can include a stimulator for application to the tongue of a user. In an aspect, the stimulator can be configured to stimulate tongue muscle spindles via stochastic resonance. Stimulating tongue muscle spindles via stochastic resonance can generate afferent nerve signals to the brain, producing a therapeutic effect. In an aspect, the stimulator can include a transducer configured to produce vibrations. In an aspect, the stimulator can further include a tongue pad configured to be coupled to the transducer. The transducer and tongue pad can be used to produce and apply vibrations to the tongue of a user to stimulate tongue muscle spindles via stochastic resonance.

In an aspect, the stimulator can further include a receiver configured to receive a signal. The receiver can enable the stimulator to communicate with an external device. In an aspect, the signal can include information about a type of the vibrations. In an aspect, the signal can be generated using an application on an electronic device in wireless communication with the receiver. While not required by the present disclosure, using an application on an external device to generate the signal can reduce the number and complexity of components of the stimulator, thus reducing the size and cost of the stimulator.

In an aspect, the stimulator can further include an amplifier configured to amplify the signal. In an aspect, the stimulator can further include a controller configured to transmit the signal to the transducer to produce the type of the vibrations. The amplifier and controller can increase the amplitude of the signal and direct the signal to produce vibrations capable of stimulating tongue muscle spindles.

In an aspect, the type of the vibrations can be at least one of Brownian, pink, or white noise. Implementing a type of vibrations with stochastic properties such as Brownian, pink, or white noise can generate stochastic resonance among the nerves that innervate the tongue, causing them to send afferent signals back to the brain.

In an aspect, the signal can further include information about a frequency range of the vibrations. In an aspect, the frequency range can be within about 1 Hz to about 300 Hz. The frequency range can include frequencies likely to cause tongue muscle spindles to generate nerve signals in response to the vibrations. In an aspect, the type of the vibrations can be Brownian noise.

In an aspect, the receiver can be a personal area network (PAN) receiver.

In an aspect, the transducer can be an electromagnetic acoustic transducer. In another aspect, the transducer can be a piezoelectric transducer. While not required by the present disclosure, implementing a piezoelectric transducer can improve power output and signal response and broaden available output frequency ranges.

In an aspect, the tongue pad can be configured to be in contact with a top surface of the tongue. The tongue pad being in contact with a top surface of the tongue can provide vibrations transmitted through the tongue pad access to tongue muscle spindles.

In an aspect, the tongue pad can be configured to be detachable from a body portion of the stimulator. In an aspect, the tongue pad can be one of a plurality of interchangeable tongue pads. Implementing a plurality of detachable, interchangeable tongue pads can provide for the tongue pads fitting different portions of a user's tongue, or user tongues of various sizes and structures.

In an aspect, the electronic device can be a mobile phone. Implementing a mobile phone can provide a user with a convenient platform to operate the stimulator.

In an aspect, the type of the vibrations can be selectable by a user between at least one of Brownian, pink, or white noise. The type of the vibrations being selectable between at least one of Brownian, pink, or white noise can enable a user to select a type of vibrations associated with increased therapeutic effects.

Aspects of a method of treatment according to the present disclosure can include selecting a type of vibrations to be at least one of Brownian, pink, or white noise. Selecting a type of vibrations with stochastic properties such as Brownian, pink, or white noise can generate stochastic resonance among the nerves that innervate the tongue, causing them to send afferent signals back to the brain. In an aspect, the method of treatment can further include selecting a frequency range of the vibrations. A user can select a frequency range associated with increased therapeutic effects.

In an aspect, the method of treatment can further include applying a stimulator to the tongue of a user to apply the vibrations to the tongue of the user. In an aspect, applying the stimulator to the tongue of the user can stimulate tongue muscle spindles via stochastic resonance. Stimulating tongue muscle spindles via stochastic resonance can generate afferent nerve signals to the brain, producing a therapeutic effect. In an aspect, the stimulator can include a transducer configured to produce the vibrations. In an aspect, the stimulator can further include a tongue pad coupled to the transducer. The transducer and tongue pad can be used to produce and apply vibrations to the tongue of a user to stimulate tongue muscle spindles via stochastic resonance.

In an aspect, the frequency range of the vibrations can be within about 1 Hz to about 300 Hz. For example, in an aspect, the frequency range of the vibrations can be within about 20 Hz to about 250 Hz. In an aspect, the frequency range of the vibrations can be within about 1 Hz to about 150 Hz. The frequency range can include frequencies likely to cause tongue muscle spindles to generate nerve signals in response to the vibrations.

In an aspect, the type of the vibrations can be Brownian noise. In another aspect, the type of the vibrations can be pink noise. The type of vibrations can be selected to be Brownian noise or pink noise when vibrations with greater intensity at lower frequencies are desired.

In an aspect, the method of treatment can further include applying the stimulator to the tongue of the user for a time period within about 5 minutes to about 30 minutes. For example, in an aspect, the time period can be about 10 minutes to about 25 minutes. Applying the stimulator to the tongue of the user for a time period within about 5 minutes to about 30 minutes can provide great enough stimulation of tongue muscle spindles to achieve a therapeutic effect.

In an aspect, a post-treatment severity of a symptom of the user can be less than about 75% of the pre-treatment severity of the symptom. For example, in an aspect, a post-treatment severity of a symptom of the user can be less than about 50% of the pre-treatment severity of the symptom.

### BRIEF DESCRIPTION OF THE DRAWINGS/FIGURES

The accompanying figures, which are incorporated herein and form a part of the specification, illustrate the present disclosure and, together with the description, further serve to explain the principals thereof and to enable a person skilled in the pertinent art to make and use the same. Aspects of the present disclosure are best understood from the following detailed description when read with the accompanying figures. It is noted that features may not be drawn to scale. In fact, the dimensions of the features may be arbitrarily increased or reduced for clarity of discussion. In the drawings:

FIG. 1A is a side view of a stimulator device in use, according to an aspect.

FIG. 1B is a side view of a stimulator device in use, according to an aspect.

FIG. 2A is a top view of the stimulator device shown in FIG. 1, according to an aspect.

FIG. 2B is a bottom view of the stimulator device shown in FIG. 1, according to an aspect.

FIG. 2C is a front view of the stimulator device shown in FIG. 1, according to an aspect.

FIG. 2D is a perspective view of the stimulator device shown in FIG. 1, according to an aspect.

FIG. 3A is an exploded view of the stimulator device shown in FIG. 1, according to an aspect.

FIG. 3B is an exploded view of the stimulator device shown in FIG. 1, according to an aspect.

FIG. 4 is an exploded view of a tongue pad and coupling device, according to an aspect.

FIGS. 5A-5B are perspective views of tongue pads, according to an aspect.

FIG. 6 is a block diagram illustrating a configuration of the stimulator device shown in FIG. 1, according to an aspect.

FIG. 7 is a block diagram illustrating a configuration of the stimulator device shown in FIG. 1 and an electronic device, according to an aspect.

FIG. 8 is an illustration of the stimulator device illustrated in FIG. 6 connected to the electronic device illustrated in FIG. 7, according to an aspect.

FIG. 9 is an illustration of an interface for communicating with the stimulator device shown in FIG. 1 and illustrated in FIG. 6, according to an aspect.

FIG. 10 is an illustration of the stimulator device illustrated in FIG. 6 connected to the electronic device illustrated in FIG. 8 and a server, according to an aspect.

FIG. 11 is a block diagram illustrating a configuration of the treatment device shown in FIG. 1, according to an aspect.

FIG. 12 is a flowchart illustrating the process of treating an individual using the stimulator device illustrated in FIG. 6 or 11.

FIG. 13 is an illustration of a patient questionnaire, according to an aspect.

FIG. 14 is a graph illustrating treatment results, according to an aspect.

FIG. 15 is a block diagram illustrating a configuration of an example computer system useful for implementing various aspects of the present disclosure.

### DETAILED DESCRIPTION

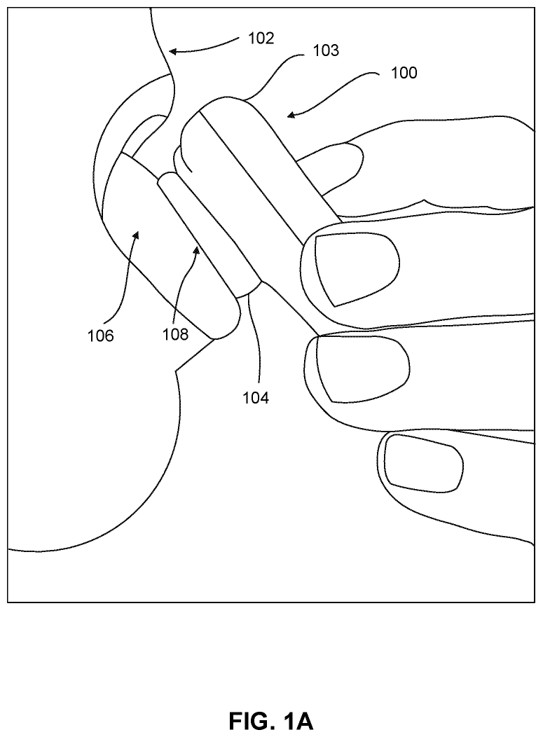

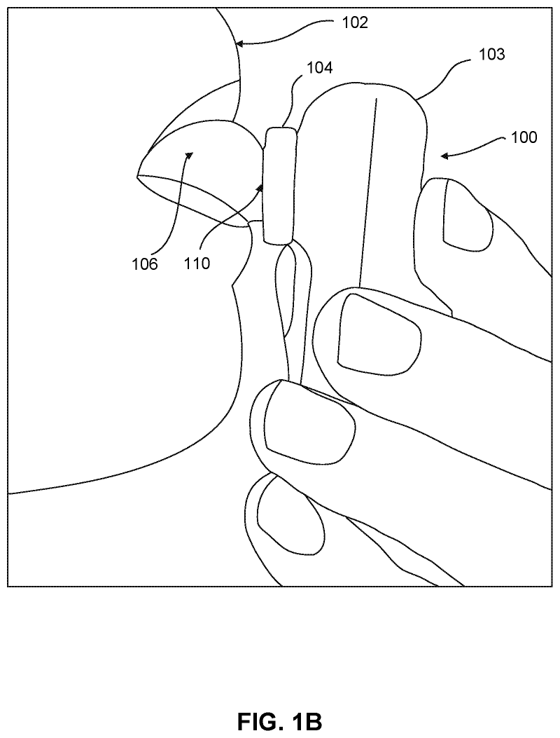

FIG. 1A shows a stimulator device 100 in use on a user 102. Stimulator device 100 can be configured to apply vibrations to a user 102's tongue 106. Stimulator device 100 can include a body portion 103. Body portion 103 can contain the internal components of stimulator device 100 described herein.

Stimulator device 100 can also include a tongue pad 104. In an aspect, tongue pad 104 can be configured to contact a top surface of tongue 106. In another aspect, tongue pad 104 can be configured to contact a side or bottom surface of tongue 106. Tongue pad 104 can be configured to vibrate against a surface of tongue 106. The vibrations can be produced using components within body portion 103 of stimulator device 100, as discussed below.

In an aspect, tongue pad 104 can be detachable from body portion 103. In another aspect, tongue pad 104 can be integral with body portion 103. In an aspect, tongue pad 104 can protrude from body portion 103, as shown in FIGS. 1A and 1B. In another aspect, tongue pad 104 can be integrated within body portion 103 and not substantially protrude from body portion 103. In another aspect, stimulator device 100 can include no tongue pad 104 (i.e., body portion 103 can be configured to contact and vibrate against a surface of tongue 106 directly, in place of tongue pad 104). When the application of tongue pad 104 to tongue 106 is discussed below, it should be understood that each of these configurations can be implemented.

In an aspect, user 102 or another individual can grip body portion 103 to apply tongue pad 104 to user 102's tongue 106, thus applying vibrations through tongue pad 104 to tongue 106. In an aspect, tongue pad 104 can be applied to the top surface of a middle portion 108 of user 102's tongue 106, as shown in FIG. 1A. In another aspect, tongue pad 104 can be applied to the top surface of the tip 110 of user 102's tongue 106, as shown in FIG. 1B.

As shown in FIGS. 1A and 1B, in an aspect, stimulator device 100 can be applied to user 102's tongue 106 without stimulator device entering user 102's mouth. This can maximize user comfort during use. In another aspect, stimulator device 100 can applied to user 102's tongue while inserting stimulator device 100 into user 102's mouth.

Vibrations produced using stimulator device 100 and passed through tongue pad 104 can be a type of vibrations which has stochastic properties (i.e., includes a random assortment of frequencies). For example, vibrations produced by stimulator device 100 can include at least one of Brownian, pink, or white noise.

In using the terms “Brownian (or “brown”), “pink,” or “white” noise, it should be understood that the “color” of noise refers to the power spectrum (i.e., the distribution of intensity as a function of spatial frequency) of a noise signal. The power spectrum is also known as the power spectral density, and has units of watts (W)/hertz (Hz) or decibel-milliwatts (dBm)/Hz. “Brownian noise,” also referred to as “red noise” or “brown noise,” has a power spectral density that decreases proportionally to 1/f². In practice, this means that Brownian noise loses intensity as frequency increases, in comparison, more quickly than do pink or white noise. “Pink noise” has a power spectral density that decreases proportionally to 1/f, whereas white noise has a constant power spectral density across frequencies.

Additionally, “blue” and “violet” noise will be discussed in the following disclosure. “Blue noise” has a power spectral density that increases proportionally to f, while “violet noise” has a power spectral density that increases proportionally to f². Therefore, violet noise gains intensity as frequency increases, in comparison, more quickly than does blue noise.

Since intensity decreases or increases (or stays static) with increasing frequency at different rates across the above noise colors, different noise colors can correspond to different physiological effects when vibrations following the power spectral density of a particular noise color are applied to the body. This can result from the fact that nerves (e.g., sensory neurons within mechanoreceptors) within the body are configured to fire at certain threshold stimulation inputs. Further, these threshold inputs can be dependent upon frequency of stimulation. Therefore, nerves that are more susceptible to firing in the presence of lower-frequency stimulations can be more susceptible to firing in the presence of Brownian noise, which exhibits higher intensities at lower frequencies. Comparatively, nerves that are more susceptible to firing in the presence of higher-frequency stimulations can be more susceptible to firing in the presence of white noise, which exhibits equal intensities across all frequencies.

Nerves within tongue muscle spindles can be optimally stimulated by vibrations in a frequency range within about 1 Hz to about 150 Hz. However, tongue muscle spindles can be stimulated by vibrations in a frequency range within about 1 Hz to about 300 Hz. Additionally, other therapeutic effects can be achieved by implementing vibrations in a frequency range including frequencies above those corresponding to optimal tongue muscle spindle stimulation. For example, vibrations in a frequency range within about 1 Hz to about 20 KHz can be implemented using stimulator device 100. Regardless of the frequency range of the implemented vibrations, it can be important to include vibrations having frequencies at which tongue muscle spindles are stimulated (e.g., about 1 Hz to about 300 Hz).

In an aspect, stimulator device 100 can be configured to stimulate tongue muscle spindles via stochastic resonance. For example, stochastic vibrations applied to tongue 106 of user 102 using stimulator device 100 can predispose nerves within the tongue muscle spindles (e.g., sensory neurons within mechanoreceptors) to fire (i.e., generate nerve signals). This is due to the inclusion of a wide range of frequencies within the vibrations. Input signals that would ordinarily be too weak to cause a nerve to generate an output signal can resonate with a vibration signal of a given frequency within the vibrations. This can cause the input signal to be enhanced, thus effectively lowering the nerve's firing threshold and causing the nerve to fire. Therefore, the generation of nerve signals can be increased by applying vibrations with stochastic properties to a surface of a user 102's tongue.

Increased generation of nerve signals can have therapeutic effects. For example, when sensory neurons within mechanoreceptors generate nerve signals, the flow of sensory information traveling between the peripheral nervous system (PNS), which includes sensory neurons within the tongue, and the central nervous system (CNS) can be increased. Increasing the flow of sensory information between the PNS and CNS can restore normal levels of sensory and motor information exchange, aiding the restoration of normal bodily function. As a non-limiting example, increased neural activity within the PNS can contribute to the formation of new sensorimotor pathways that aid in developing muscle stability, enhancing muscle strength, and reducing pain by addressing proprioceptive deficiencies.

Therefore, stimulator device 100 can be applied to tongue 106 of a user to achieve the benefits associated with increased generation of nerve signals, for example, pain reduction.

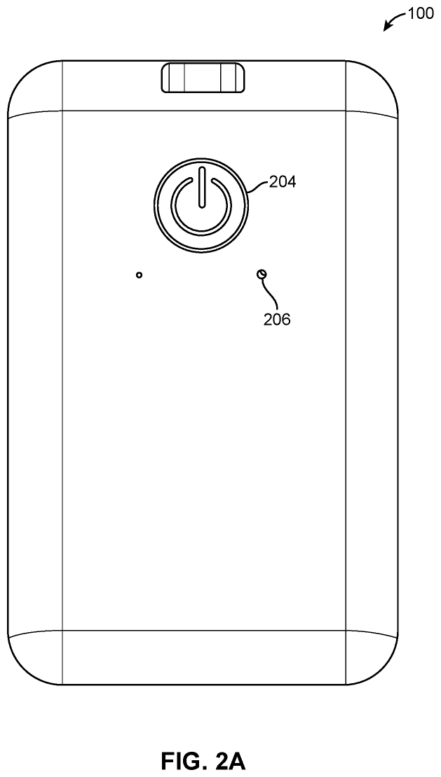

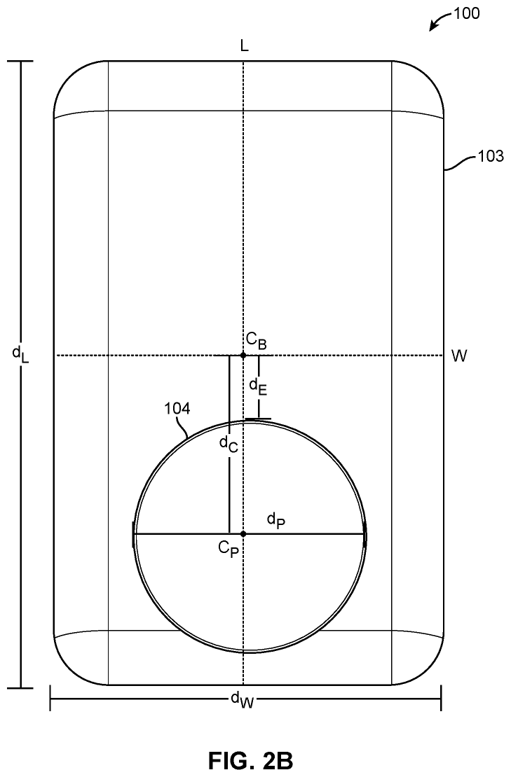

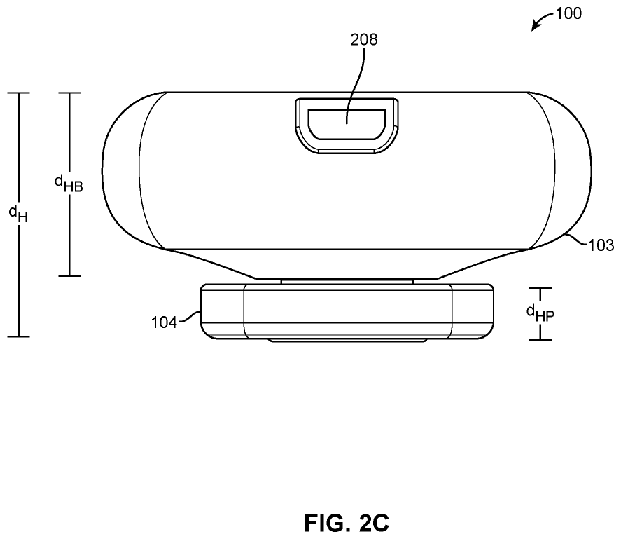

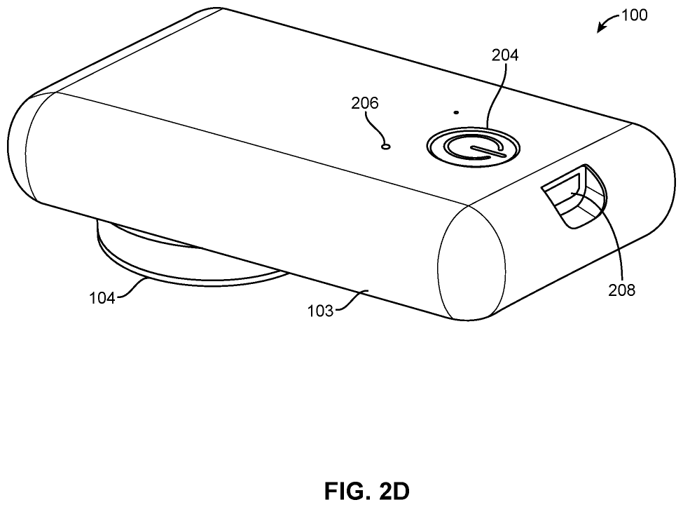

FIG. 2A shows a top of stimulator device 100. As shown in FIG. 2A, stimulator device 100 can include an on/off switch 204 to control the power state of stimulator device 100. In an aspect, on/off switch 204 can be a push-button switch. In another aspect, on/off switch 204 can be a slide switch or other suitable switch. In another aspect, stimulator device 100 can be remotely powered on or off via a connected external device.

Stimulator device can also include an indicator 206 to indicate the status of stimulator device 100. For example, indicator 206 can indicate whether stimulator device 100 is on or off. Additionally or alternatively, indicator 206 can indicate whether stimulator device 100 is connected to an external device (e.g., via a personal area network (PAN)). In an aspect, indicator 206 can be a light (e.g., an LED). In another aspect, indicator 206 can be an e-ink display. In an aspect, indicator 206 can indicate the status of stimulator device 100 via changing patterns of flashing. For example, indicator 206 can flash to indicate that stimulator device 100 is powered on and cease flashing when stimulator device 100 has connected to an external device. In another aspect, indicator 206 can indicate the status of stimulator device 100 by changing color. The use of indicator 206 can be accompanied by auditory signals further indicating the status of stimulator device 100.

FIG. 2B shows a bottom of stimulator device 100. As shown in FIG. 2B, stimulator device can have a long axis (L) and a short axis (W). Further, body portion 103 of stimulator device 100 can have a center point C_{B}, though which axes L and W can pass. Tongue pad 104 can have a center point C_{P}.

The length (d_{L}) of stimulator device 100 can be about 4 cm to about 16 cm. Specifically, d_{L} can be about 5 cm to about 14 cm, about 6 cm to about 12 cm, or about 7 cm to about 10 cm. The width (d_{W}) of stimulator device 100 can be about 2 cm to about 7 cm. Specifically, d_{W} can be about 3 to about 6 cm or about 4 to about 5 cm. The ratio of d_{W}/d_{L} can be about ⅛ to about 1. Specifically, d_{W}/d_{L} can be about ⅙ to about ⅛, about ¼ to about ¾, or about ⅓ to about ⅝.

In an aspect, tongue pad 104 can be centered on axis L, as shown in FIG. 2B. In another aspect, tongue pad 104 can be offset from axis L. In an aspect, tongue pad 104 can be centered on axis W. In another aspect, tongue pad 104 can be offset from axis W, as shown in FIG. 2B. For example, a distance (d_{C}) between center point C_{B} of body portion 103 and center point C_{P} of tongue pad 104 can be about 0.1 cm to about 10 cm. Specifically, d_{C} can be about 0.5 cm to about 8 cm, about 1 cm to about 5 cm, or about 2 cm to about 3 cm.

In an aspect, C_{P} can be approximately equidistant between C_{B} and the edge of stimulator device 100 along axis L. In another aspect, C_{P} can be closer to an edge of stimulator device 100 along axis L than to C_{b}.

The diameter (d_{P}) of tongue pad 104 can be about 1 cm to about 5 cm. Specifically, d_{P} can be about 1.5 cm to about 4 cm, about 1.5 to about 3.5 cm, or about 2 to about 3 cm. The diameter (d_{P}) of tongue pad 104 and its positioning can be such that a distance d_{E} extends between an edge of tongue pad 104 and center point C_{B} of body portion 103 along axis L. The ratio of d_{E}/d_{C} can be about 0 to about ¾. Specifically, d_{E}/d_{C} can be about ¼ to about ¾, about ¼ to about ½, or about ¼ to about ⅓.

FIG. 2C shows a front of stimulator device 100. The height (d_{H}) of stimulator device 100 can be about 0.5 cm to about 4 cm. Specifically, d_{H} can be about 0.75 cm to about 3.75 cm, about 1.25 cm to about 3.25 cm, or about 1.75 cm to about 2.75 cm. The height (d_{HB}) of body portion 103 of stimulator device 100 can be about 0.35 cm to about 3.85 cm. Specifically, d_{HB} can be about 1 cm to about 3 cm, about 1.25 cm to about 2.75 cm, or about 1.5 cm to about 2 cm. The height (d_{HP}) of tongue pad 104 can be about 0.15 cm to about 3.65 cm. Specifically, d_{HP} can be about 0.2 cm to about 2.5 cm, about 0.3 cm to about 1.5 cm, or about 0.4 cm to about 0.75 cm. The ratio of d_{HB}/d_{H} can be about ½ to about 11/12. Specifically, d_{HB}/d_{H} can be about ½ to about 9/10, about ⅔ to about ⅛, or about ¾ to about ⅘. The ratio of d_{HP}/d_{H} can be about 1/12 to about ½. Specifically, d_{HP}/d_{H} can be about ⅛ to about ½, about ⅙ to about ⅓, or about ⅕ to about ¼.

The dimensions of stimulator device 100 can be selected such that stimulator device 100 can fit comfortably within user 102's mouth while providing tongue pad 104 access to tongue 106. Further, the dimensions of stimulator device 100 can be selected such that stimulator device 100 can be grasped and controlled comfortably with a single hand of user 102.

As shown in FIG. 2C, stimulator device 100 can include a port 208. Port 208 can be a charging port. In addition, port 208 can be a port for exchanging data between stimulator device 100 and an external device. In an aspect, port 208 can be a micro USB port. In another aspect, port 208 can be a USB Type C port. For example, port 208 can be configured for charging stimulator device 100 using a 5V micro USB or a 5V, 9V, 12V, 15V, or 20V USB Type C charger.

FIG. 2D shows stimulator device 100, including body portion 103, tongue pad 104, on/off switch 204, indicator 206, and port 208. As shown in FIG. 2D, at least one of on/off switch 204 or indicator 206 can be disposed on an opposite side of stimulator device 100 from tongue pad 104 such that on/off switch 204 and/or indicator 206 can be visible and accessible while stimulator device 100 is in use.

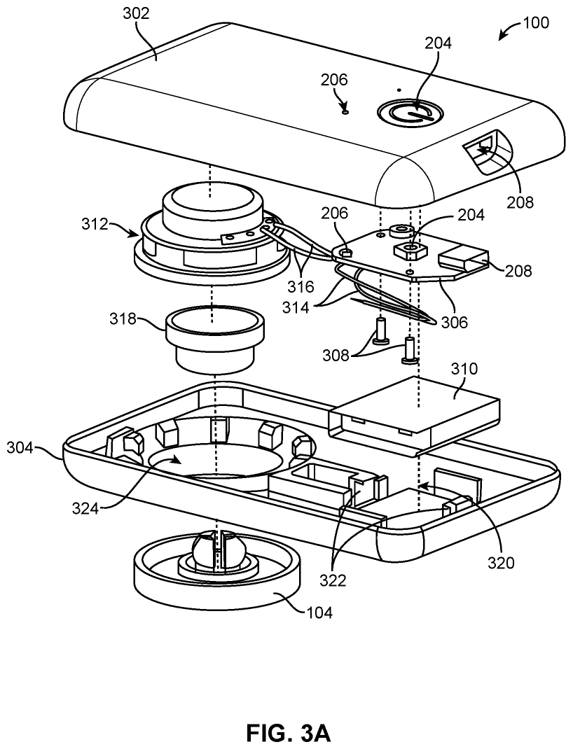

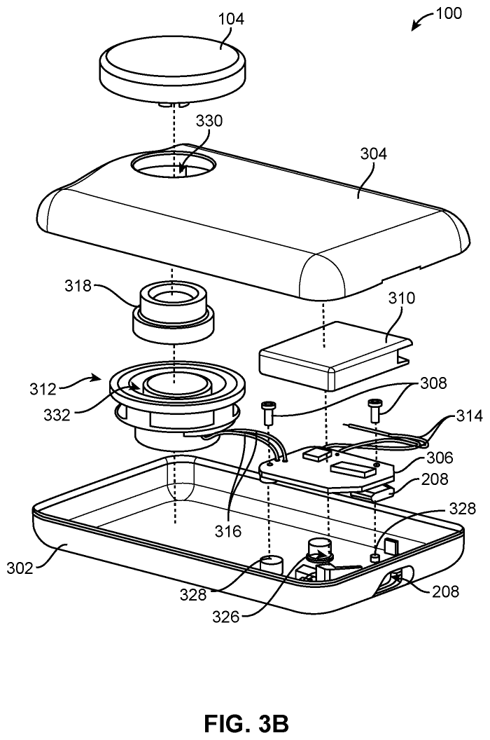

FIG. 3A shows stimulator device 100 in an exploded view. As shown in FIG. 3A, stimulator device 100 can include a top housing 302 and a bottom housing 304 to house various components of stimulator device 100. Top housing 302 and bottom housing 304 can be formed using any suitable polymer. For example, top housing 302 and bottom housing 304 can be 3D printed using a white powder based polyamide such as PA 2200 or PA 2201, or other polymer suitable for 3D printing. Additionally, top housing 302 and bottom housing 304 can be injection molded using a thermoplastic polymer such as acrylonitrile butadiene styrene (ABS), nylon polyamide (NA), polyethylene (PE), polypropylene (PP), polystyrene (PS), or other polymer suitable for injection molding.

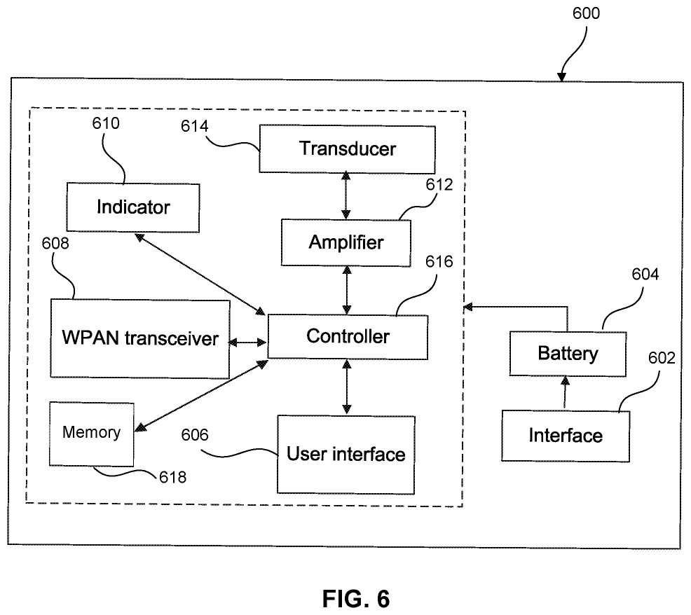

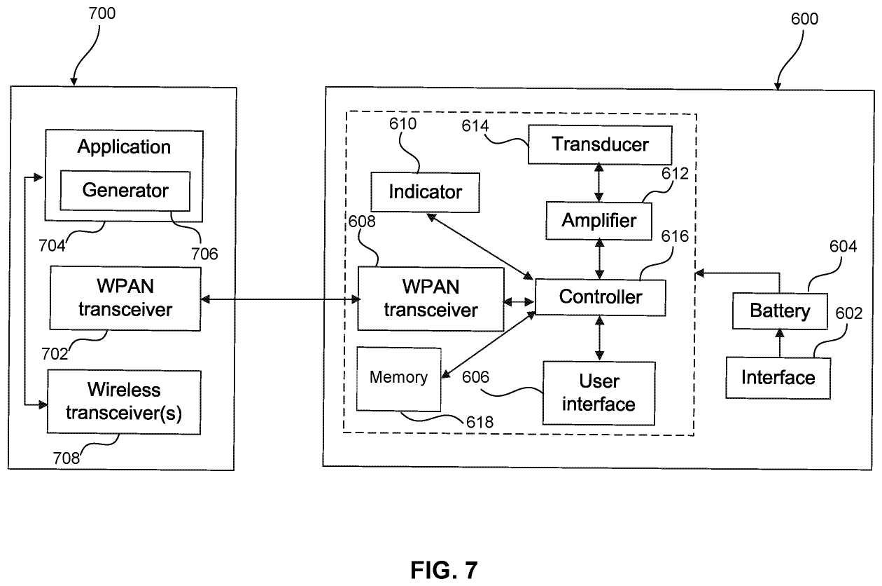

A printed circuit board (PCB) 306 can be housed within top housing 302 and bottom housing 304. PCB 306 can connect various electronic components of stimulator device 100, as shown in FIGS. 6 and 7. For example, PCB 306 can include at least one of on/off switch 204, indicator 206, or port 208. In an aspect, PCB 306 can be attached to top housing 302. In another aspect, PCB 306 can be attached to bottom housing 304.

Stimulator device 100 can also include fasteners 308 to attach PCB 306 to top housing 302 or bottom housing 304. In an aspect, fasteners 308 can be screws. In another aspect, fasteners can be glue or another adhesive.

Stimulator device 100 can also include a battery 310 for supplying power to stimulator device 100. In an aspect, battery 310 can be a rechargeable battery. In another aspect, battery 310 can be a replaceable battery or batteries. Specifically, battery 310 can be a DC 3.7V/200 mAh, 3.7V/500 mAh, or a 3.7V/700 mAh rechargeable battery. Battery 310 can be selected such that a time stimulator device 100 can be operated (i.e., vibrations can be produced by stimulator device 100) is at least 2 hours. Specifically, the time stimulator device 100 can be operated can be from about 2 to about 24 hours, about 2 to about 12 hours, about 2 to about 6 hours, or about 2 to about 3 hours. Additionally, battery 310 can be selected such that it can be fully charged within about an hour or less using, for example, a 5V micro USB or a 5V, 9V, 12V, 15V, or 20V USB Type C charger. Specifically, battery 310 can be fully charged within about 5 minutes to about an hour, about 20 minutes to about an hour, or about 45 minutes to about an hour.

Stimulator device can also include a transducer 312 to produce vibrations. Transducer 312 can convert electrical signals into vibrations to further generate pressure waves (e.g., sound). In an aspect, transducer 312 can be an electromagnetic acoustic transducer (EMAT). In such an aspect, transducer 312 can have an impedance of about 2, about 4, about 6, or about 8 ohms (Ω) and a wattage of about 1, about 2, about 3, about 4, or about 5 watts (W).

In another aspect, transducer 312 can be a piezoelectric transducer. Implementing a piezoelectric transducer can provide at least one of enhanced power output, improved signal response, or broader output frequency ranges. In such an aspect, to ensure transducer 312 can provide sufficient displacement to produce vibrations which stimulate tongue muscle spindles, transducer 312 can be a piezoelectric stack actuator including multiple piezoelectric elements. Piezoelectric elements arranged in a stack can multiply the linear displacement produced when an electric field is applied to the stack of piezoelectric elements, as compared to a single piezoelectric element. Accordingly, linear displacement along a major axis of a piezoelectric stack actuator can be about 5 μm to about 80 μm. Additionally or alternatively, transducer 312 can be an amplified piezoelectric actuator. For example, transducer 312 can be a piezoelectric stack actuator amplified using a lever arm. A lever arm can be used to amplify and translate displacement along the major axis of a piezoelectric stack actuator to displacement along a minor axis of the piezoelectric stack actuator. The displacement of the lever arm along the minor axis can be about 10 times to about 40 times the displacement of the piezoelectric stack actuator along the major axis, providing sufficient displacement for producing vibrations which stimulate tongue muscle spindles.

Transducer 312 can range in size from about 10 mm to about 60 mm. Specifically, transducer 312 can range in size from about 15 mm to about 50 mm, about 20 mm to about 40 mm, or about 25 mm to about 35 mm. Transducer 312 can be capable of producing vibrations with a frequency range within about 20 Hz to about 20 kHz. For example, transducer 312 can be a 28 mm 423 W speaker capable of producing vibrations with a frequency range within 20 Hz to 20 kHz. However, transducers capable of producing vibrations with a frequency range within a broader frequency range, for example, about 1 Hz to about 20 kHz, are contemplated.

The type, size, and power output of transducer 312 can be selected to increase a treatment effect, for example, by producing more powerful vibrations with a larger and more complete frequency range. While FIG. 3A shows a single transducer 312, additional transducers, such as two, three, or four transducers 312, can be included in stimulator device 100.

Stimulator device 100 can also include connection wires 314 to connect components on PCB 306 to battery 310. Additionally, stimulator device 100 can include connection wires 316 to connect components on PCB 306 to transducer 312. The connection and operation of various components within stimulator device 100 will be described in more detail with respect to FIGS. 6, 7, and 11.

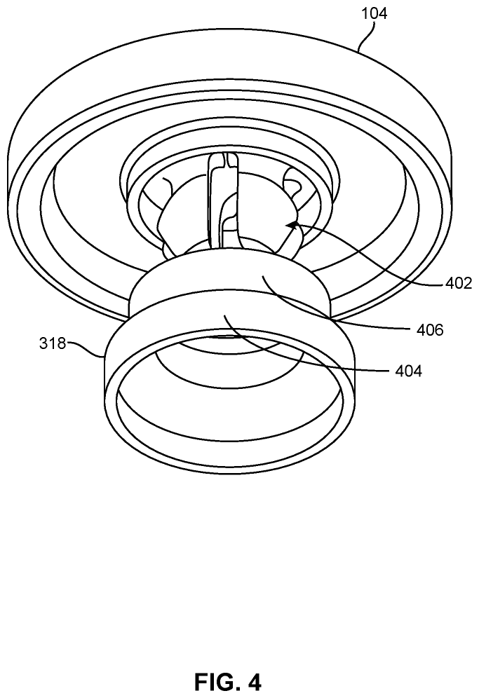

Stimulator device 100 can also include ring 318 to couple transducer 312 to tongue pad 104. Ring 318 can be formed using the same polymer material as that of top housing 302 and bottom housing 304, or a different material. The coupling of transducer 312 to tongue pad 104 using ring 318 will be described in more detail with respect to FIGS. 3B and 4.

Stimulator device 100 can also include a first recess 320 to receive battery 310. In an aspect, first recess 320 can be disposed in bottom housing 304. In another aspect, first recess 320 can be disposed in top housing 302. Stimulator device 100 can also include hooks 322 to secure battery 310 to top housing 302 or bottom housing 304. Hooks 322 can be integrally formed with top housing 302 or bottom housing 304.

Stimulator device 100 can also include a second recess 324 to receive transducer 312 and ring 318. In an aspect, second recess 324 can be disposed in bottom housing 304. In another aspect, second recess 324 can be disposed in top housing 302.

FIG. 3B shows stimulator device 100 in an exploded view. As shown in FIG. 3B, stimulator device 100 can also include a mounting region 326 to receive and mount PCB 306. In an aspect, mounting region 326 can be disposed on top housing 302. In another aspect, mounting region 326 can be disposed on bottom housing 304. Mounting region 326 can include holes 328 to receive fasteners 308. Holes 328 can be threaded holes to receive fasteners 308 when fasteners 308 are screws.

Stimulator device 100 can also include aperture 330 to receive ring 318. Aperture 330 receiving ring 318 can make ring 318 accessible for the coupling of tongue pad 104 to ring 318, described in more detail with respect to FIG. 4.

To couple tongue pad 104 to transducer 312, ring 318 can first be coupled to transducer 312. For example, in an aspect, the housing of transducer 312 can include a channel 332 to receive ring 318. In an aspect, channel 332 can be an annular channel configured to couple ring 318 to transducer 312 via an interference fit. In an aspect, channel 332 can be a threaded annular channel configured to couple ring 318 to transducer 312 via a threaded interference fit. In such an aspect, ring 318 can be outfitted with corresponding threads. Additionally, an adhesive or other fastener can be used to couple ring 318 to transducer 312, complementing an interference fit. In another aspect, ring 318 can be coupled to transducer 312 purely using an adhesive or other fastener. In another aspect, ring 318 can be integrally formed with the housing of transducer 312.

FIG. 4 shows the coupling of tongue pad 104 to ring 318. As shown in FIG. 4, tongue pad 104 can include prongs 402. Prongs 402 can be formed using a flexible material. For example, prongs 402 can be formed using a material with a flexural modulus of about 500 Pa to about 2000 Pa. Specifically, prongs 402 can be formed using a material with a flexural modulus of about 700 Pa to about 1800 Pa, about 900 Pa to about 1600 Pa, or about 1100 Pa to about 1400 Pa. Prongs 402 can be 3D printed using a white powder based polyamide such as PA 2200 or PA 2201, or other polymer suitable for 3D printing. Additionally, prongs 402 can be injection molded using a thermoplastic polymer such as acrylonitrile butadiene styrene (ABS), nylon polyamide (NA), polyethylene (PE), polypropylene (PP), polystyrene (PS), or other polymer suitable for injection molding. In an aspect, prongs 402 can be formed using the same material as that of tongue pad 104 (i.e., they can be integrally formed). In another aspect, prongs 402 can be formed using a different material. Prongs 402 can widen as they extend from tongue pad 104.

Ring 318 can include a base portion 404 and an upper portion 406 to receive prongs 402. For example, as shown in FIG. 4, an inner diameter of base portion 404 can be smaller than an inner diameter of upper portion 406 such that prongs 402 can be inserted into ring 318 through upper portion 406 and secured within ring 318 via a snap fit. A user can couple tongue pad 104 to ring 318 by aligning prongs 402 with the lumen of upper portion 406 and pressing downward, and can detach tongue pad 104 from ring 318 by pulling tongue pad 104 away from ring 318. While not shown in FIG. 4, when a user is attaching/detaching tongue pad 104 to/from ring 318, ring 318 can be coupled to transducer 312 and can be included within body portion 103 of stimulator device 100.

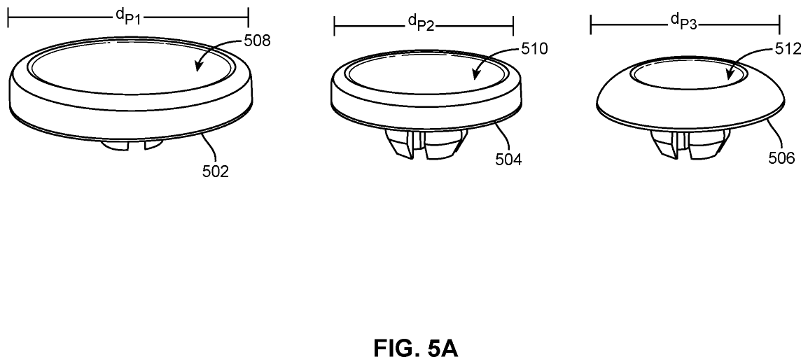

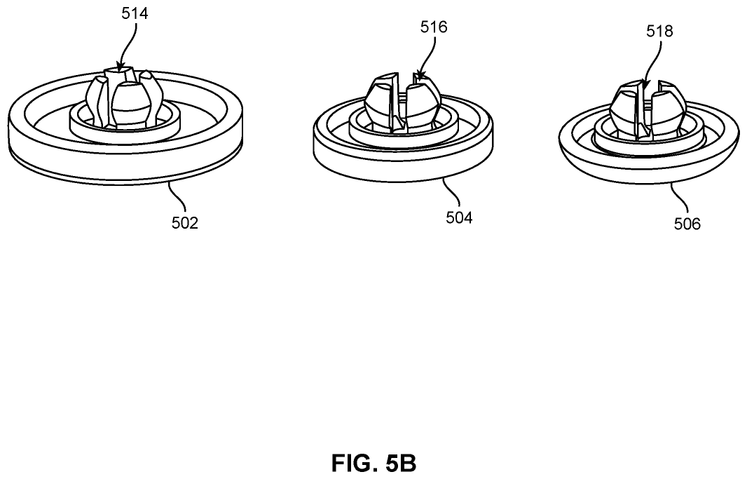

FIG. 5A shows a plurality of tongue pads for use with stimulator device 100. FIG. 5A shows a first tongue pad 502, a second tongue pad 504, and a third tongue pad 506. First, second, and third tongue pads 502, 504, 506 can be 3D printed using a white powder based polyamide such as PA 2200 or PA 2201, or other polymer suitable for 3D printing. Additionally, first, second, and third tongue pads 502, 504, 506 can be injection molded using a thermoplastic polymer such as acrylonitrile butadiene styrene (ABS), nylon polyamide (NA), polyethyelene (PE), polypropylene (PP), polystyrene (PS), or other polymer suitable for injection molding. In an aspect, first, second, and third tongue pads 502, 504, 506 can be formed using the same material as that of top housing 302 and bottom housing 304. In another aspect, first, second, and third tongue pads 502, 504, 506 can be formed using a different material.

As shown in FIG. 5A, first, second, and third tongue pads 502, 504, 506 can have different diameters d_{P1}, d_{P2}, and d_{P3}, respectively. In an aspect, first, second, and third tongue pads 502, 504, 506 can each include a depression 508, 510, and 512, respectively. Depressions 508, 510, 512 can be configured to assist a user in positioning first, second, or third tongue pads 502, 504, 506 on the user's tongue by providing tactile feedback to the user (i.e., the user can feel the location of the depression). As shown in FIG. 5A, a depression can be shallow (i.e., have a depth less than about ⅕ of the height (d_{HP}) of a tongue pad, such as depression 508) or deep (i.e., have a depth equal to or greater than about ⅓ of the height (d_{HP}) of a tongue pad, such as depressions 510 and 512). Additionally, a depression can be shaped as a spherical cap (such as depressions 508, 510, and 512) or cylindrically shaped. The surface of a tongue pad can also include convex portions (such as for third tongue pad 506). In another aspect, at least one of depressions 508, 510, or 512 can be replaced entirely with a convex or flat surface such that at least one of first, second, or third tongue pads 502, 504, 506 does not include a depression.

While FIG. 5A shows circular first, second, and third tongue pads 502, 504, 506, first, second, and third tongue pads 502, 504, 506 can be shaped differently. For example, at least one of first, second, or third tongue pads 502, 504, 506 can be rectangular, oval, elliptical, or any other shape. In such an aspect, d_{P1}, d_{P2}, and d_{P3} can describe the width of first, second, and third tongue pads 502, 504, 506 at their widest points.

Diameters d_{P1}, d_{P2}, d_{P3} and the shape of first, second, and third tongue pads 502, 504, 506 (including, for example, the shape of depressions 508, 510, 512) can be configured such that, collectively, first, second, and third tongue pads 502, 504, 506 can fit different portions of a user's tongue, or user tongues of various sizes and structures. For example, third tongue pad 506 can be configured to stimulate the top surface of the tip of a user's tongue, while first and second tongue pads 502, 504 can be configured to stimulate the top surface of a middle portion of the user's tongue. First and second tongue pads 502, 504 can be broader than third tongue pad 506 to contact a wider area of the user's tongue, or to provide for a user having a broader tongue. The use of a broader tongue pad, such as first tongue pad 502, can distribute vibrations such that nerves peripheral to the tongue's surface, such as the Vagus nerve innervating the palatoglossus muscle, can be stimulated.

First, second, and third tongue pads 502, 504, 506, or similar tongue pads, can be included with body portion 103 when stimulator device 100 is sold. While FIG. 5A shows three tongue pads, fewer or additional tongue pads, such as zero, one, two, four, five, or six tongue pads, can be included when stimulator device 100 is sold.

FIG. 5B shows first, second, and third tongue pads 502, 504, 506. As shown in FIG. 5B, first, second, and third tongue pads 502, 504, 506 can each include prongs 514, 516, and 518, respectively. Prongs 514, 516, 518 can be substantially the same as prongs 402, described above. Additionally, prongs 514, 516, 518 can be substantially the same size and shape such that first, second, and third tongue pads 502, 504, 506 are interchangeable within stimulator device 100. That is, a user can detach one of first, second, or third tongue pads 502, 504, 506 from body portion 103 of stimulator device 100 and replace it with another of first, second, or third tongue pads 502, 504, 506.

FIG. 6 shows components that can be included in a stimulator device 600. Stimulator device 600 can perform substantially the same functions and include similar features as stimulator device 100, described above with respect to FIGS. 1-5.

As shown in FIG. 6, in an aspect, stimulator device 600 can include an interface 602. Interface 602 can be a charging interface. In addition, interface 602 can be an interface for exchanging data between stimulator device 600 and an external device. In an aspect, interface 602 can be a port, for example, a mini USB, micro USB, or USB Type-C port or other suitable port. Specifically, interface 602 can be configured for charging stimulator device 600 using a 5V micro USB or a 5V, 9V, 12V, 15V, or 20V USB Type C charger. In another aspect, interface 602 can be a wireless charging interface. For example, interface 602 can be a Qi wireless receiver or other suitable wireless charging receiver. Interface 602 can be disposed on PCB 306.

Stimulator device 600 can also include a battery 604 for supplying power to stimulator device 600. Battery 604 can be connected to interface 602 to receive power from interface 602. In an aspect, battery 604 can be a rechargeable battery. Specifically, battery 604 can be a DC 3.7V/200 mAh, 3.7V/500 mAh, or a 3.7V/700 mAh rechargeable battery. Battery 604 can be selected such that a time stimulator device 600 can be operated (i.e., vibrations can be produced by stimulator device 600) is at least 2 hours. Specifically, the time stimulator device 600 can be operated can be from about 2 to about 24 hours, about 2 to about 12 hours, about 2 to about 6 hours, or about 2 to about 3 hours. Additionally, battery 604 can be selected such that it can be fully charged within about an hour or less using, for example, a 5V micro USB or a 5V, 9V, 12V, 15V, or 20V USB Type C charger. Battery 604 can be fully charged within about 5 minutes to about an hour, about 20 minutes to about an hour, or about 45 minutes to about an hour, using any appropriate interface 602.

Stimulator device 600 can also include a user interface 606. In an aspect, user interface 606 can be at least one of a mechanical button or switch. In another aspect, user interface 606 can be an LCD display (such as an LED display). In another aspect, user interface 606 can be an e-ink display. User interface 606 can perform at least one of the following functions: turn stimulator device 600 on and off, depict a level of remaining battery life of stimulator device 600, depict a type of vibration (e.g., at least one of Brownian, pink, or white noise) being produced by stimulator device 600, depict a frequency range of the vibrations being produced by stimulator device 600, depict a treatment duration, or depict a remaining treatment duration. User interface 606 can be disposed on PCB 306.

Stimulator device 600 can also include a wireless personal area network (PAN) transceiver 608 to receive a signal from an external device. In an aspect, WPAN transceiver 608 can be a Bluetooth transceiver or receiver. For example, WPAN transceiver 608 can be a Bluetooth V5.0+EDR (Enhanced Data Rate) transceiver. In another aspect, WPAN transceiver 608 can be a ZigBee transceiver or receiver. In another aspect, WPAN transceiver 608 can be an infrared transceiver or receiver. WPAN transceiver 608 can be disposed on PCB 306.

Stimulator device 600 can also include an indicator 610 to indicate the status of stimulator device 600. For example, indicator 610 can indicate whether stimulator device 600 is on or off. Additionally or alternatively, indicator 610 can indicate whether stimulator device 600 is connected to an external device (e.g., via WPAN transceiver 608). In an aspect, indicator 610 can be a light (e.g., an LED). In another aspect, indicator 610 can be an e-ink display. Indicator 610 can indicate the status of stimulator device 600 via changing patterns of flashing. For example, indicator 610 can flash to indicate that stimulator device 600 is powered on and cease flashing when stimulator device 600 has connected to an external device via WPAN transceiver 608. In another aspect, indicator 610 can indicate the status of device 600 by changing color. The use of indicator 610 can be accompanied by auditory signals further indicating the status of stimulator device 600. In an aspect, indicator 610 can be separate from user interface 606. In another aspect, indicator 610 can be integrated into user interface 606. Indicator 610 can be disposed on PCB 306.

Stimulator device 600 can also include an amplifier 612 to drive a transducer 614. Amplifier 612 can receive a signal (e.g., an analog audio signal), amplify the signal, and feed the amplified signal to transducer 614. (In an aspect in which amplifier 612 receives a digital audio signal, amplifier 612 can optionally include or be connected to a digital-to-analog converter (DAC) to convert the signal to an analog audio signal prior to feeding the amplified signal to transducer 614. Alternatively, transducer 614 can produce vibrations from an unconverted, digital audio signal.) Amplifier 612 can have a power output of about 1, about 2, about 3, about 4, or about 5 watts (W). For example, amplifier 612 can have a power output of 2 W. In an aspect, amplifier 612 can be a class D audio amplifier. For example, amplifier 612 can be a MAX4295 mono, 2 W, switch mode audio amplifier; a MAX9701 1.3 W, filterless, stereo audio amplifier; or another suitable class D audio amplifier. In another aspect, amplifier 612 can be a class AB audio amplifier. For example, amplifier 612 can be a MAX98309 mono, 1.4 W audio amplifier; a MAX98310 mono, 1.4 W audio amplifier; or another suitable class AB audio amplifier. In another aspect, amplifier 612 can be a class G audio amplifier. For example, amplifier 612 can be a MAX9730 2.4 W, single-supply audio amplifier or another suitable class G audio amplifier. In an aspect, amplifier 612 can be disposed on PCB 306. In another aspect, amplifier 612 can be integrated into transducer 612.

Transducer 614 can be configured to produce vibrations. Transducer 614 can convert electrical signals received from amplifier 612 into vibrations to further generate pressure waves (e.g., sound). In an aspect, transducer 614 can be an electromagnetic acoustic transducer (EMAT). In such an aspect, transducer 614 can have an impedance of about 2, about 4, about 6, or about 8 ohms ((2) and a wattage of about 1, about 2, about 3, about 4, or about 5 watts (W).

In another aspect, transducer 614 can be a piezoelectric transducer. Implementing a piezoelectric transducer can provide at least one of enhanced power output, improved signal response, or broader output frequency ranges. In such an aspect, to ensure transducer 614 can provide sufficient displacement to produce vibrations which stimulate tongue muscle spindles, transducer 614 can be a piezoelectric stack actuator including multiple piezoelectric elements. Piezoelectric elements arranged in a stack can multiply the linear displacement produced when an electric field is applied to the stack of piezoelectric elements, as compared to a single piezoelectric element. Accordingly, linear displacement along a major axis of a piezoelectric stack actuator can be about 5 μm to about 80 μm. Additionally or alternatively, transducer 614 can be an amplified piezoelectric actuator. For example, transducer 614 can be a piezoelectric stack actuator amplified using a lever arm. A lever arm can be used to amplify and translate displacement along the major axis of a piezoelectric stack actuator to displacement along a minor axis of the piezoelectric stack actuator. The displacement of the lever arm along the minor axis can be about 10 times to about 40 times the displacement of the piezoelectric stack actuator along the major axis, providing sufficient displacement for producing vibrations which stimulate tongue muscle spindles.

Transducer 614 can range in size from about 10 mm to about 60 mm. Specifically, transducer 614 can range in size from about 15 mm to about 50 mm, about 20 mm to about 40 mm, or about 25 mm to about 35 mm. Transducer 614 can be capable of producing vibrations with a frequency range within about 20 Hz to about 20 kHz. For example, transducer 614 can be a 28 mm 423 W speaker capable of producing vibrations with a frequency range within 20 Hz to 20 kHz. However, transducers capable of producing vibrations with a frequency range within a broader frequency range, for example, about 1 Hz to about 20 kHz, are contemplated.

The type, size, and power output of transducer 614 can be selected to increase a treatment effect, for example, by producing more powerful vibrations with a larger and more complete frequency range. While FIG. 6 shows a single amplifier 612 and a single transducer 614, additional amplifiers and transducers, such as two, three, or four amplifiers 612 and transducers 614, can be included in stimulator device 600.

Stimulator device 600 can also include a controller 616 to receive and transmit data within stimulator device 600. As shown in FIG. 6, in an aspect, controller 616 can transmit and receive signals to/from at least one of user interface 606, WPAN transceiver 608, indicator 610, amplifier 612, or transducer 614 via amplifier 612. In another aspect, controller 616 can communicate directly with transducer 614. Controller 616 can be disposed on PCB 306.

In an aspect, stimulator device 600 can also include memory 618 to store data within stimulator device 600. For example, in an aspect, memory 618 can store one or more files, for example, audio files. In an aspect, the one or more files can include information about a type of vibrations to be produced. For example, the one or more files can include information that the type of the vibrations is at least one of Brownian, pink, or white noise. Additionally, the one or more files can include information about a frequency range of the vibrations. For example, the one or more files can include information that the frequency range is within about 1 Hz to about 20 kHz. Specifically, the one or more files can include information that the frequency range is within about 1 Hz to about 10 kHz, within about 1 Hz to about 5 kHz, within about 1 Hz to about 1 kHz, within about 1 Hz to about 500 Hz, within about 1 Hz to about 300 Hz, within about 1 Hz to about 150 Hz, within about 12 Hz to about 300 Hz, within about 12 Hz to about 250 Hz, within about 12 Hz to about 200 Hz, within about 12 Hz to about 150 Hz, within about 20 Hz to about 300 Hz, within about 20 Hz to about 250 Hz, within about 20 Hz to about 200 Hz, or within about 20 Hz to about 150 Hz.

Memory 618 can be disposed on or connected to PCB 306. As shown in FIG. 6, controller 616 can communicate with memory 618 to retrieve and use data from the one or more files. For example, controller 616 can retrieve information on a type and frequency range of vibrations to be produced and transmit the information as a signal to amplifier 612 and transducer 614 to produce the type and frequency range of the vibrations.

In an aspect, the one or more files can be stored in memory 618 after being downloaded via WPAN transceiver 608. In another aspect, the one or more files can be recorded in memory 618 after being transferred via interface 602. In another aspect, the one or more files can be pre-loaded onto memory 618 before memory 618 is installed in stimulator device 600.

In an aspect, memory 618 can include flash memory comprising NOR and/or NAND flash memory. In an aspect, memory 618 can be internal memory. In another aspect, memory 616 can be external memory, for example, a flash drive or SD card. While these types of memory are provided as non-limiting examples, memory 618 can include any type of memory suitable for storing and accessing audio files. Memory 618 can include a memory controller that can communicate with or form a part of controller 616.

The inclusion of memory 618 in FIG. 6 should not be construed to mean that memory 618 is the only memory in electronic device 600. Other forms of secondary or primary memory can be included in electronic device 600 to perform the functions described herein.

As an example function that can be performed by stimulator device 600, user interface 606 can be used to power on stimulator device 600. Controller 616 can receive a signal from user interface 606 indicating the status of stimulator device 600 has changed from powered off to powered on. Controller 616 can transmit a signal to indicator 610 instructing indicator 610 to indicate stimulator device 600 has been powered on. For example, in an aspect in which indicator 610 is an LED, indicator 610 can receive an instruction from controller 616 to turn on and optionally begin flashing. In an aspect in which indicator 610 is integrated into user interface 606, controller 616 can transmit a single to user interface 606 instructing user interface 606 to display a powered on status. In addition, controller 616 can transmit a signal to transducer 614 instructing transducer 614 to play a sound indicating stimulator device 600 has been powered on.

As an additional example function that can be performed by stimulator device 600, WPAN transceiver 608 can receive a signal from an external device. WPAN transceiver 608 can then transmit a signal indicating connection to an external device to controller 616. Controller 616 can receive the signal and transmit a signal to indicator 610 instructing indicator 610 to indicate connection to an external device. For example, in an aspect in which indicator 610 is an LED, indicator 610 can receive an instruction from controller 616 to at least begin or cease flashing, change color, or turn off. In an aspect in which indicator 610 is integrated into user interface 606, controller 616 can transmit a single to user interface 606 instructing user interface 606 to display a connected status. In addition, controller 616 can transmit a signal to transducer 614 instructing transducer 614 to play a sound indicating stimulator device 600 is connected to an external device. A similar function can be performed to indicate stimulator device 600 has been disconnected from an external device.

As an additional example function that can be performed by stimulator device 600, WPAN transceiver 608 can receive a signal. In an aspect, the signal can include an analog audio signal. In another aspect, the signal can include a digital audio signal. WPAN transceiver can then transmit the signal to controller 616. Controller 616 can receive the signal and transmit the signal to amplifier 612 (optionally including or connected to a DAC). Amplifier 612 can receive and amplify the signal (optionally employing the DAC to convert the signal to an analog audio signal). Amplifier 612 can then transmit the signal to transducer 614, which can produce vibrations in accordance with the signal. The signal can include information about a type of vibrations to be produced. For example, the signal can include information that the type of the vibrations is at least one of Brownian, pink, or white noise. Additionally, the signal can include information about a frequency range of the vibrations. For example, the signal can include information that the frequency range is within about 1 Hz to about 20 kHz. Specifically, the signal can include information that the frequency range is within about 1 Hz to about 10 kHz, within about 1 Hz to about 5 kHz, within about 1 Hz to about 1 kHz, within about 1 Hz to about 500 Hz, within about 1 Hz to about 300 Hz, within about 1 Hz to about 150 Hz, within about 12 Hz to about 300 Hz, within about 12 Hz to about 250 Hz, within about 12 Hz to about 200 Hz, within about 12 Hz to about 150 Hz, within about 20 Hz to about 300 Hz, within about 20 Hz to about 250 Hz, within about 20 Hz to about 200 Hz, or within about 20 Hz to about 150 Hz.

As shown in FIG. 6, battery 604 can supply power to at least one of user interface 606, WPAN transceiver 608, indicator 610, amplifier 612, or transducer 614 to perform the above exemplary functions.

It should be understood that FIG. 6 shows an example configuration of components within stimulator device 600. The components shown in FIG. 6 can connected in alternative arrangements to accomplish the objectives of the present disclosure. Further, components can be omitted, and components other than or in addition to those specified can be employed to accomplish the objectives of the present disclosure. For example, while not shown in FIG. 6, stimulator device 600 can also include a wireless local area network (WLAN) transceiver to facilitate connection of stimulator device 600 to networks, for example, Wi-Fi networks.

FIG. 7 shows stimulator device 600 connected to an external electronic device 700. Electronic device 700 can be a portable electronic device. For example, electronic device 700 can be a mobile phone, smart watch or other wearable, tablet, laptop computer, desktop computer, personal digital assistant (PDA), MP3 player, etc.

As shown in FIG. 7, electronic device 700 can include a wireless personal area network (WPAN) transceiver 702. WPAN transceiver 702 can transmit and receive signals to/from WPAN transceiver 608 of stimulator device 600. In an aspect, WPAN transceiver 702 can be a Bluetooth transceiver or transmitter. For example, WPAN transceiver 702 can be a Bluetooth V5.0+EDR (Enhanced Data Rate) transceiver. In another aspect, WPAN transceiver 702 can be a ZigBee transceiver or transmitter. In another aspect, WPAN transceiver 702 can be an infrared transceiver or transmitter.

Further, an application 704 can be run on electronic device 700. Application 704 can run a generator 706 to generate signals. For example, generator 706 can generate audio signals (e.g., analog or digital audio signals). (In an aspect in which generator 706 generates analog audio signals, electronic device 700 can optionally include an analog-to-digital converter (ADC) to convert the signals to digital audio signals prior to transmitting the signals to stimulator device 600 via WPAN transceiver 702. Alternatively, electronic device can transmit an unconverted, analog signal.) Using application 704, a user can select a type of vibrations to be encoded in a signal produced by generator 706. For example, a user can select a type of vibrations to be at least one of Brownian, pink, or white noise. Further, using application 704, a user can select a frequency range of the vibrations to be encoded in the signal produced by generator 706. For example, a user can select a frequency range of the vibrations to be within about 1 Hz to about 20 KHz. Specifically, a user can select a frequency range of the vibrations to be within about 1 Hz to about 10 kHz, within about 1 Hz to about 5 kHz, within about 1 Hz to about 1 kHz, within about 1 Hz to about 500 Hz, within about 1 Hz to about 300 Hz, within about 1 Hz to about 150 Hz, within about 12 Hz to about 300 Hz, within about 12 Hz to about 250 Hz, within about 12 Hz to about 200 Hz, within about 12 Hz to about 150 Hz, within about 20 Hz to about 300 Hz, within about 20 Hz to about 250 Hz, within about 20 Hz to about 200 Hz, or within about 20 Hz to about 150 Hz. Generator 706 can encode at least one of the type of the vibrations or frequency range of the vibrations into a signal (e.g., an analog or digital audio signal).

In an aspect, application 704 can be an application designed specifically for use with stimulator device 600 or other similar stimulator devices. In another aspect, application 704 can be a general use noise generator application. For example, application 704 can be the “Noise Generator” application produced by TMSOFT, or another similar application, available for download from the Apple App Store, Google Play Store, or another similar platform. However, it should be understood that when application 704 is a general use noise generator application, some of the functionality specific to use with stimulator device 600 discussed below (e.g., receiving a treatment recommendation) may be unavailable to a user.

Electronic device 700 can also include wireless transceiver(s) 708. Wireless transceiver(s) 708 can include at least one of a wireless wide area network (WWAN) transceiver, a wireless local area network (WLAN) transceiver, or a wireless metropolitan area network (WMAN) transceiver. Transceiver(s) 708 can be used to connect to networks, for example, at least one of cellular or Wi-Fi networks, or any suitable network.

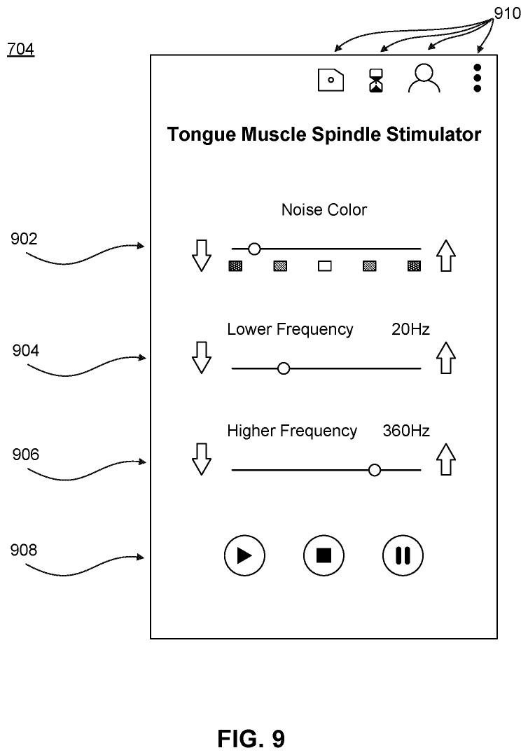

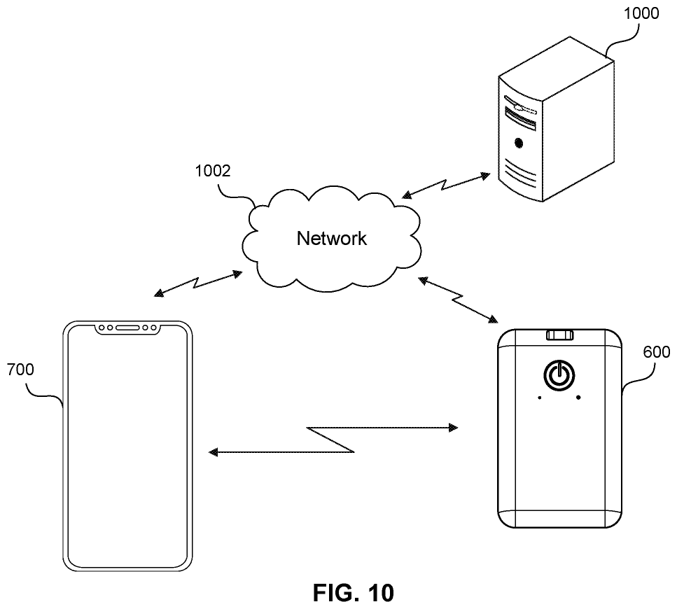

As an example function that can be performed by electronic device 700, application 704 can collect and transmit data including information on at least one of treatment vibration type(s), treatment frequency range(s), or treatment duration for a treatment session or sessions to wireless transceiver(s) 708. Application 704 can also collect and transmit data including information input by a user (described in more detail with respect to FIGS. 9 and 10) to transceiver(s) 708. Wireless transceiver(s) 708 can transmit the data over a network to an external device and/or server.

As an additional example function that can be performed by electronic device 700, application 704 can be used select a type of vibrations. For example, application 704 can be used to select a type of vibrations to be at least one of Brownian, pink, or white noise. Application 704 can further be used to select a frequency range of the vibrations. For example, application 704 can be used to select a frequency range of the vibrations to be within about 1 Hz to about 20 kHz. Specifically, application 704 can be used to select a frequency range of the vibrations to be within about 1 Hz to about 10 kHz, within about 1 Hz to about 5 kHz, within about 1 Hz to about 1 kHz, within about 1 Hz to about 500 Hz, within about 1 Hz to about 300 Hz, within about 1 Hz to about 150 Hz, within about 12 Hz to about 300 Hz, within about 12 Hz to about 250 Hz, within about 12 Hz to about 200 Hz, within about 12 Hz to about 150 Hz, within about 20 Hz to about 300 Hz, within about 20 Hz to about 250 Hz, within about 20 Hz to about 200 Hz, or within about 20 Hz to about 150 Hz. Generator 706 on application 704 can encode at least one of the type of the vibrations or frequency range of the vibrations into a signal (e.g., an analog or digital audio signal). Application 704 can transmit the signal to WPAN transceiver 702. WPAN transceiver 702 can transmit the signal to WPAN transceiver 608 of stimulator device 600.

In another aspect, additionally or alternatively, application 704 can be used to select the type and frequency range of the vibrations by providing an interface to select one or more files stored in memory 618 of stimulator device 600 for stimulator device 600 to play (e.g., using transducer 614). In such an aspect, application 704 may not include generator 706 and/or the vibration type and frequency range controls described with respect to FIG. 9. In an aspect, volume controls on electronic device 700 can be used to control the volume at which stimulator device 600 produces vibrations.

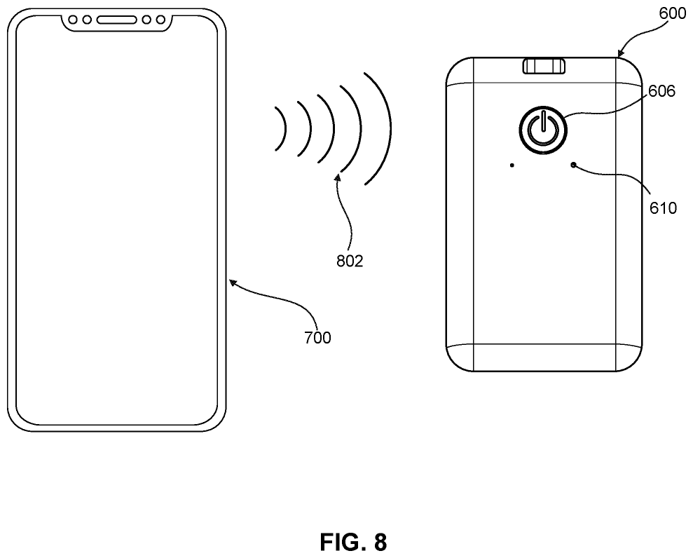

FIG. 8 shows stimulator device 600 connected to electronic device 700. As shown in FIG. 8, a signal 802 can be transmitted from electronic device 700 to stimulator device 600. In an aspect, signal 802 be transmitted over a personal area network (PAN). For example, signal 802 can be transmitted using Bluetooth, ZigBee, or infrared. As noted above, signal 802 can include information about a type of vibrations to be produced by stimulator device 600. Signal 802 can also include information about a frequency range of the vibrations.

As shown in FIG. 8, user interface 606 of stimulator device 600 can be an on-off switch. When a user employs user interface 606 to power on stimulator device 600 in range of electronic device 700, electronic device can detect the presence of stimulator device 600 via a wireless PAN. Electronic device 700 can send stimulator device 600 a request to connect to stimulator device 600 on the PAN. Once stimulator device 600 receives the connection request and is connected to electronic device 700, indicator 610 can be updated to indicate connection to electronic device 700, as discussed above.

FIG. 9 shows an example application 704 for use on electronic device 700. As shown in FIG. 9, application 704 can include a sound color selector 902 to select a type of vibrations to be produced by stimulator device 600. Sound color selector 902 can be used to select at least one of Brownian, pink, or white noise. As shown in FIG. 9, sound color selector 902 can be used to select additional types of vibrations, such as at least one of blue or violet noise. Additionally, sound color selector 902 can be used to select combinations of two types of vibrations, for example, Brownian-pink noise, as shown in FIG. 9. (As discussed above, Brownian noise can also be referred to as “red” noise. Therefore, FIG. 9 shows Brownian noise with a red color code.)

Application 704 can also include a frequency minimum selector 904 and a frequency maximum selector 906. Frequency minimum selector 904 can be used to select a minimum frequency of a frequency range of the vibrations to be produced by stimulator device 600. For example, frequency minimum selector 904 can be used to select a minimum frequency within a range of about 1 Hz to about 150 Hz. Specifically, frequency minimum selector 904 can be used to select a minimum frequency of about 1 Hz, about 12 Hz, about 20 Hz, about 50 Hz, about 100 Hz, or about 150 Hz. Frequency maximum selector 906 can be used to select a maximum frequency of a frequency range of the vibrations to be produced by stimulator device 600. For example, frequency maximum selector 906 can be used to select a maximum frequency within a range of about 20 Hz to about 20 kHz. Specifically, frequency maximum selector 906 can be used to select a maximum frequency of about 20 Hz, about 50 Hz, about 100 Hz, about 150 Hz, about 200 Hz, about 300 Hz, about 500 Hz, about 1 kHz, about 5 kHz, about 10 kHz, or about 20 kHz. In an aspect, frequency minimum selector 904 and frequency maximum selector 906 can be separate controls, as shown in FIG. 9. In another aspect, frequency minimum selector 904 and frequency maximum selector 906 can be combined, such as in a single, sliding scale frequency range control.

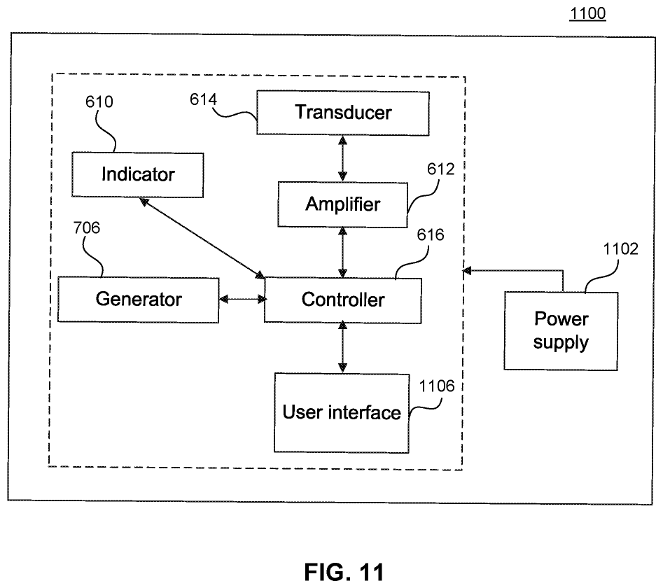

Application 704 can also include a start/stop control 908. Start stop/control 908 can be used to start and stop vibrations produced by stimulator device 600 once at least one of a type of the vibrations or frequency range of the vibrations has been selected. When a treatment session is conducted, start/stop control 908 can be used to start, stop, and/or pause the treatment session. In an aspect, application 704 can include start/stop control 908 while not including sound color selector 902, frequency minimum selector 904, and frequency maximum selector 906, such that application 704 can be used to start and stop an audio file already stored on stimulator device 600, as discussed with respect to FIGS. 6 and 11.

Application 704 can also include treatment profile controls 910. Treatment profile controls 910 can be used to perform at least one of the following functions: set a treatment timer, record a duration of a treatment, save treatment settings (i.e., vibration type and frequency range), or preserve user-recorded treatment data. Via treatment profile controls 910, a user can open and record treatment notes to preserve data related to a treatment session or multiple treatment sessions. For example, a user can record information including at least one of an approximate severity of symptoms pre-treatment (e.g., on a 0-10 scale), the type(s) of symptom(s) experienced, an approximate severity of symptom(s) post-treatment (e.g., on a 0-10 scale), or any changes in the type(s) of symptom(s) experienced. A user can also track at least one of a change in symptom type(s) and/or symptom(s) severity across multiple treatment sessions. Such user-recorded data can be attached to data collected by application 704, which can include information on at least one of treatment duration, vibration type(s) used during treatment session(s), or vibration frequency ranges used during treatment session(s). As noted above, data recorded by a user and/or application 704 can be transmitted by electronic device 700 to an external device and/or server, which will be described in more detail with respect to FIG. 10.

Additionally, application 704 can be used to provide a treatment recommendation to a user. The treatment recommendation can include at least one of a recommended vibration type (e.g., at least one of Brownian, pink, or white noise), a recommended frequency range, or a recommended treatment duration. The treatment recommendation can be based on at least one of a type of symptom or a severity of a symptom, which can be input by a user via treatment profile controls 910. For example, a user can input that the user has been experiencing light and sound sensitivity. A user can also input that the severity of the light and sound sensitivity has been about an 8 on a 0-10 scale (in which 0 indicates no symptoms and 10 indicates debilitating symptoms). Based on at least one of the identified symptom type (light and sound sensitivity) or the symptom severity (8 on a 0-10 scale), application 704 can recommend, for example, implementing white noise in a frequency range from 20 Hz to 150 Hz for 20 minutes.

The treatment recommendation can also be based on at least one of a plurality of symptom types or a plurality of symptom severities, which can be input by a user via treatment profile controls 910. For example, a user can input that the user has been experiencing light and sound sensitivity and a headache. A user can also input that the severity of the light and sound sensitivity has been about an 8 on a 0-10 scale (in which 0 indicates no symptoms and 10 indicates debilitating symptoms) and the severity of the headache has been about a 6 on a 0-10 scale. Based on at least one of the plurality of identified symptom types (light and sound sensitivity and headache) or the plurality of symptom severities (8 and 6 on a 0-10 scale), application 704 can recommend, for example, implementing Brownian noise in a frequency range from 20 Hz to 250 Hz for 15 minutes.

The treatment recommendation can also include a recommended plan for treatment, including, for example, a recommended frequency of treatment. For example, the treatment recommendation can include a recommendation to apply a treatment twice daily, daily, every other day, bi-weekly, weekly, or at any other suitable frequency.

The treatment recommendation can be based on treatment data collected by application 704 run on a plurality of electronic devices 700 used by a plurality users. The treatment data can include at least one of treatment vibration type(s), treatment frequency range(s), treatment duration, approximate severity of symptoms pre-treatment (e.g., on a 0-10 scale), type(s) of symptom(s) experienced, approximate severity of symptom(s) post-treatment (e.g., on a 0-10 scale), or any changes in the type(s) of symptom(s) experienced for individual treatment sessions conducted by multiple users.

FIG. 10 shows stimulator device 600 and electronic device 700 connected to a server 1000 via a network 1002. Server 1000 can store treatment data. As shown in FIG. 10, stimulator device 600 and/or electronic device 700 can communicate with server 1000 via network 1002 to exchange data, for example, treatment data and the treatment recommendation discussed above.

Treatment data can be sent to server 1000 by a plurality of electronic devices 700. The treatment data can then be analyzed at server 1000 to identify treatment recommendations for at least one of a symptom type(s) or symptom(s) severity. For example, a treatment recommendation can be generated using an algorithm that matches maximally successful treatment profiles stored at server 1000 with at least one of the symptom type(s) or symptom(s) severity input by a user. Specifically, the algorithm can identify treatment profiles (including information on at least one of vibration type(s), frequency range(s), or treatment duration) that are associated with maximally reduced symptom severity for at least one of a particular symptom type(s) or symptom(s) severity, wherein at least one of the particular symptom type(s) or symptom(s) severity matches the symptom type(s) and/or symptom(s) severity input by the user. The algorithm can then use data from the identified maximally successful treatment profiles to construct the treatment recommendation. For example, the algorithm can construct the treatment recommendation by identifying at least one of an approximate frequency range most frequently used in the maximally successful treatment profiles, a most frequently used vibration type(s) in the maximally successful treatment profiles, an approximate duration most frequently used in the maximally successful treatment profiles, or an approximate frequency of treatment used in a series of maximally successful treatment profiles.

In an aspect, the treatment recommendation can be generated on server 1000 and sent to application 704 in response to a user inputting at least one of a symptom type(s) or symptom(s) severity in application 704. In another aspect, the treatment recommendation can be generated on application 704, using data received from server 1000, in response to a user inputting at least one of a symptom type(s) or symptom(s) severity in application 704. Treatment recommendations for various symptoms and/or severities of symptoms generated on server 1000 or application 704 can be continually updated as server 1000 receives new data from users of stimulator device 600.

Network 1002 can be the Internet. Network 1002 can be wireless wide area network (WWAN), a wireless local area network (WLAN), a wireless metropolitan area network (WMAN), or any other suitable network.

While FIG. 10 shows a single server 1000, a plurality of servers 1000 can be implemented. In an aspect, stimulator device 600 can communicate with server 1000 via electronic device 700. In another aspect, stimulator device 600 can communicate with server 1000 directly, for example, to transmit treatment data to server 1000.

FIG. 11 shows components that can be included in stimulator device 1100 in an alternative configuration to stimulator device 600. Stimulator device 1100 can perform substantially the same functions as stimulator devices 100 and 600, described above with respect to FIGS. 1-10. Further, the same features of indicator 610, amplifier 612, transducer 614, and controller 616 can be included in stimulator device 1100. However, in stimulator device 1100, generator 706 can be integrated within stimulator device 1100 rather than provided by an application (though in an aspect, stimulator device 1100 can optionally interact with an application, as described below). Additionally, stimulator device 1100 can include a user interface 1106, which can include similar features as and perform some or all of the functions that can be performed by application 704 run on electronic device 700 and user interface 606 of stimulator device 600. Accordingly, in an aspect, stimulator device 1100 can be operated without interacting with an application run on an external device.

As shown in FIG. 11, stimulator device 1100 need not include an interface or battery. Stimulator device 1100's power supply 1102 can be a plug-in power supply. However, in an aspect, stimulator device 1100 can include an interface and battery such as interface 602 and battery 604 of stimulator device 600.

As an example function that can be performed by stimulator device 1100, user interface 1106 can be used select a type of vibrations. For example, user interface 1106 can be used to select a type of vibrations to be at least one of Brownian, pink, or white noise. User interface 1106 can further be used to select a frequency range of the vibrations. For example, user interface 1106 can be used to select a frequency range of the vibrations to be within about 1 Hz to about 20 kHz. Specifically, user interface 1106 can be used to select a frequency range of the vibrations to be within about 1 Hz to about 10 kHz, within about 1 Hz to about 5 kHz, within about 1 Hz to about 1 kHz, within about 1 Hz to about 500 Hz, within about 1 Hz to about 300 Hz, within about 1 Hz to about 150 Hz, within about 12 Hz to about 300 Hz, within about 12 Hz to about 250 Hz, within about 12 Hz to about 200 Hz, within about 12 Hz to about 150 Hz, within about 20 Hz to about 300 Hz, within about 20 Hz to about 250 Hz, within about 20 Hz to about 200 Hz, or within about 20 Hz to about 150 Hz. User interface 1106 can transmit a signal to generator 706 via controller 616 instructing generator 706 to encode at least one of the type of the vibrations or frequency range of the vibrations into a signal (e.g., an analog or digital audio signal). Generator 706 can transmit the signal to amplifier 612 via controller 616. Amplifier 612 can receive and amplify the signal (optionally employing the DAC to convert the signal to an analog audio signal). Amplifier 612 can then transmit the signal to transducer 614, which can produce vibrations in accordance with the signal.

In an aspect, generator 706 can include memory, for example, memory 618 described with respect to FIG. 6. Accordingly, generator 706 can store one or more files that include vibration type and/or frequency range information, which can be accessed by controller 616 to produce vibrations of a particular type and/or frequency range.

Generator 706 and user interface 1106 can have varying levels of complexity. For example, in an aspect, user interface 1106 can include only an on/off switch (e.g., on/off switch 204) that powers on/off stimulator device 1100 and starts or stops a file stored in generator 706. The file can include information on vibration type (e.g., Brownian) and frequency range of the vibrations (e.g., 20 Hz to 250 Hz). When activated via user interface 1106, stimulator device 1100 can play the file using transducer 614 at a fixed, pre-set volume to produce the vibrations. The intensity of the vibrations as experienced by a user can be adjusted by altering the pressure with which stimulator device 1100 is applied to the tongue of the user.

As a more complex example, user interface 1106 can include a touch screen, mechanical buttons, and/or capacitive sensors configured for control of one or more of: vibration type, volume, and frequency range. For example, in an aspect, user interface 1106 can include one or more capacitive sensors configured for volume and/or frequency range control. In an aspect, a user can control volume and/or frequency range by contacting the one or more capacitive sensors according to predetermined patterns. For example, a swipe upward could increase volume (by steps or continuously), while a swipe downward could decrease volume (by steps or continuously). Similarly, a swipe to the right on a portion of or one of the one or more capacitive sensors could increase the maximum of the frequency range (by steps or continuously), while a swipe to the left could decrease the maximum of the frequency range (by steps or continuously). A swipe to the right on another portion of or another of the one or more capacitive sensors could increase the minimum of the frequency range (by steps or continuously), while a swipe to the left could decrease the minimum of the frequency range (by steps or continuously). In an aspect, the one or more capacitive sensors can be coupled to PCB 306 such that contact of a user to body 103 of stimulator device 1100 can be sensed and used to control vibration parameters, as described above, without the user directly contacting the one or more capacitive sensors.

These functions and example components of user interface 1106 are providing as non-limiting examples. User interface 1106 can include a touch screen, mechanical buttons, and/or capacitive sensors for controlling any combination of vibration type, volume, and frequency range or any of these parameters alone (e.g., volume alone). In an aspect, user interface 1106 can include a touch screen for the selection of one or more files stored in generator 706 for stimulator device 1100 to play (e.g., using transducer 614).

It should be understood that FIG. 11 shows an example configuration of components within stimulator device 1100. The components shown in FIG. 11 can connected in alternative arrangements to accomplish the objectives of the present disclosure. Further, components can be omitted, and components other than or in addition to those specified can be employed to accomplish the objectives of the present disclosure. For example, while not shown in FIG. 11, in an aspect, stimulator device 1100 can also include a wireless local area network (WLAN) and/or wireless personal area network (WPAN) transceiver (such as WPAN transceiver 608) to facilitate connection of stimulator device 1100 to networks, for example, Wi-Fi or Bluetooth networks. Thus, connection of stimulator device 1100 to a server, for example server 1000 via network 1002, can be achieved. Additionally, the functions of generating and receiving a treatment recommendation, as described above, can be conducted using stimulator device 1100 and server 1000 as they can be using stimulator device 600, electronic device 700, and server 1000. Additionally, new files can be downloaded to generator 706 via the WLAN and/or WPAN transceiver. In an aspect, a user can select a new file to be downloaded, or an old file to be deleted, from an external device in communication with stimulator device 1100. For example, in an aspect, a user can select a new file to be downloaded, or an old file to be deleted, from application 704 running on electronic device 700. However, in an aspect, no WLAN and/or WPAN transceiver is included in stimulator device 1100, as shown in FIG. 11. In such an aspect, the one or more files stored in generator 706 can either be static or can be modified via an external device connected to stimulator device 1100 via a wired connection (e.g., to an interface 602).

It is to be understood that the treatment methods disclosed herein can be achieved using either the combination of stimulator device 600 and electronic device 700 (optionally with server 1000) or stimulator device 1100 (optionally with server 1000). Therefore, any discussion of treatment methods should not be construed to limit the device employed to a particular aspect. For example, some or all of the functions that can be performed on electronic device 700 can be performed on stimulator device 1100, as discussed above.

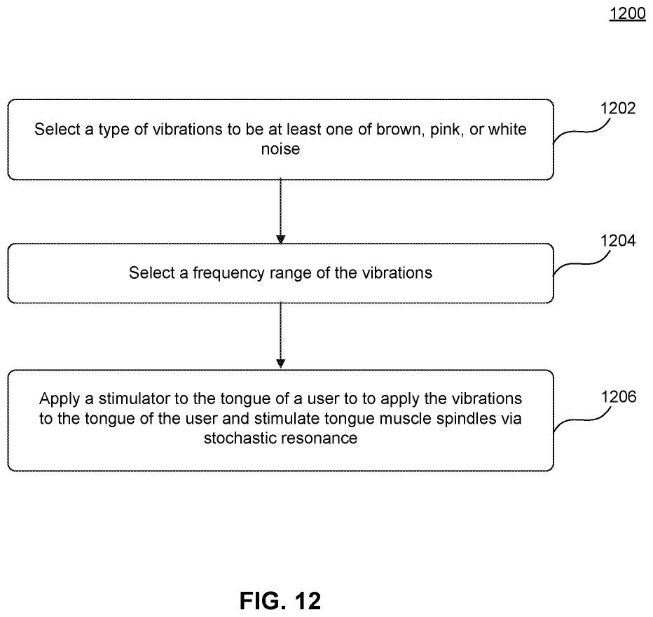

FIG. 12 shows an example method of treatment 1200 using stimulator device 600 or 1100.

Unless stated otherwise, the steps of method 1200 need not be performed in the order set forth herein. Additionally, unless specified otherwise, the steps of method 1200 need not be performed sequentially. The steps can be performed in a different order or simultaneously. Further, method 1200 need not include all the steps illustrated. As one example, method 1200 need not include step 1202, for example, if a type of vibrations has been pre-set. As another example, method 1200 need not include step 1204, for example, if a frequency range of the vibrations has been pre-set. In an aspect, the type of vibrations and/or frequency range, for example, can be pre-set by encoding a file onto memory included within stimulator device 600 or 1100 (e.g., memory 618).

In step 1202, a user can select a type of vibrations to be at least one of Brownian, pink, or white noise, according to the methods provided in the above disclosure. Additionally, in an aspect, a user can select the type of vibrations to be at least one of blue or violet noise.

In step 1204, a user can select a frequency range of the vibrations, according to the methods provided in the above disclosure. For example, a user can select a frequency range of the vibrations to be within about 1 Hz to about 20 kHz. Specifically, a user can select a frequency range to be within about 1 Hz to about 10 kHz, within about 1 Hz to about 5 kHz, within about 1 Hz to about 1 kHz, within about 1 Hz to about 500 Hz, within about 1 Hz to about 300 Hz, within about 1 Hz to about 150 Hz, within about 12 Hz to about 300 Hz, within about 12 Hz to about 250 Hz, within about 12 Hz to about 200 Hz, within about 12 Hz to about 150 Hz, within about 20 Hz to about 300 Hz, within about 20 Hz to about 250 Hz, within about 20 Hz to about 200 Hz, or within about 20 Hz to about 150 Hz.

In an aspect, the type of vibrations and/or frequency range of the vibrations can be selected using an application (e.g., application 704) operated on an external device (e.g., electronic device 700). In an aspect, the type of vibrations and/or frequency range of the vibrations can be selected using a user interface (e.g., user interface 1106) of a stimulator device (e.g., stimulator device 1100). In an aspect, the type of vibrations and/or frequency range of the vibrations can be selected by selecting a file for a stimulator device (e.g., stimulator device 600 or 1100) to play, the file being stored on the stimulator device or an external electronic device (e.g., electronic device 700) and selected via an application (e.g., application 704) operated on the external electronic device or a user interface (e.g., user interface 1106) of the stimulator device.

In step 1206, a user can apply a stimulator to the tongue of a user (either oneself or another user) to apply the vibrations to the tongue of the user and stimulate tongue muscle spindles via stochastic resonance, according to the methods provided in the above disclosure. For example, the stimulator can be a stimulator device according to the aspects described above for stimulator device 600 and stimulator device 1100. A user can apply the stimulator to the tongue of the user for a time period within about 5 minutes to about 30 minutes. Specifically, the time period can be about 10 minutes to about 25 minutes, about 15 minutes to about 20 minutes, or about 15 minutes. The time period can be either continuous or segmented. For example, the user can repeatedly apply the stimulator for time segments of about 2 to about 5 minutes, with rest in between the time segments, until a total time period of application reaches a time period as disclosed above.

Prior to conducting a full treatment session, a user can repeat steps 1202 through 1206, adjusting vibration type and frequency range settings in steps 1202 and 1204, until the user experiences immediate comfort when applying the stimulator to the tongue in step 1206. A user experiencing immediate comfort can be an indicator that the treatment session will be effective. However, immediate user comfort is not required to predict the effectiveness of a treatment session.

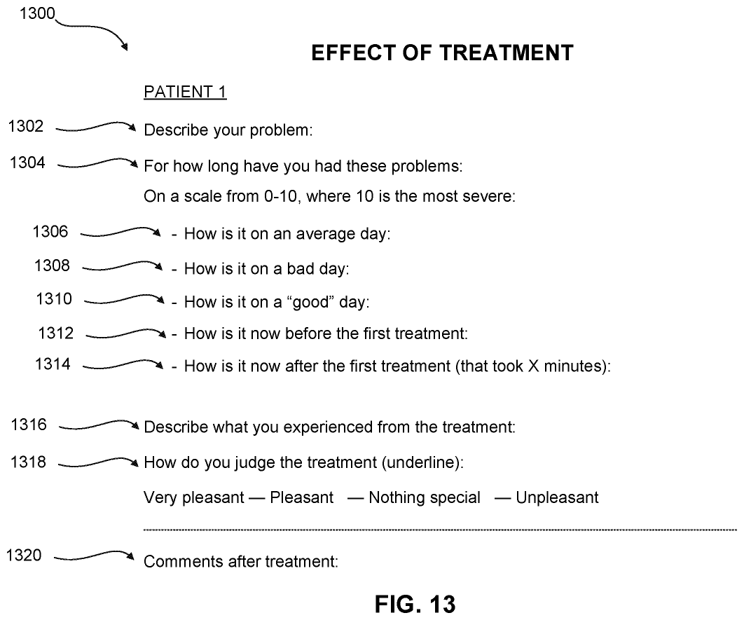

FIG. 13 shows an example patient questionnaire 1300. Patient questionnaire 1300 can be used to evaluate treatment effects for a user. For example, patient questionnaire 1300 can be administered to a user before and after treatment. In an aspect, patient questionnaire can be administered to a user by another individual. In another aspect, patient questionnaire can be administered to a user via application 704. Further, in addition to or in place of the treatment data discussed above, user responses to elements of patient questionnaire 1300 can be included in the data recorded and/or sent by electronic device 700 to server 1000.

Patient questionnaire can include prompts for at least one of a symptom(s) description 1302, a symptom(s) duration 1304, an average symptom(s) severity 1306, a worst symptom(s) severity 1308, a best symptom(s) severity 1310, a pre-treatment symptom(s) severity 1312, a post-treatment symptom(s) severity 1314, a treatment results description 1316, a treatment characterization 1318, or post-treatment comments 1320. For symptom(s) severity (prompts 1306-1314), a scale of 0-10 can be used, where 0 indicates no symptoms and 10 indicates debilitating severity (i.e., an individual has difficulty performing basic functions such as walking, talking, eating, or sleeping).

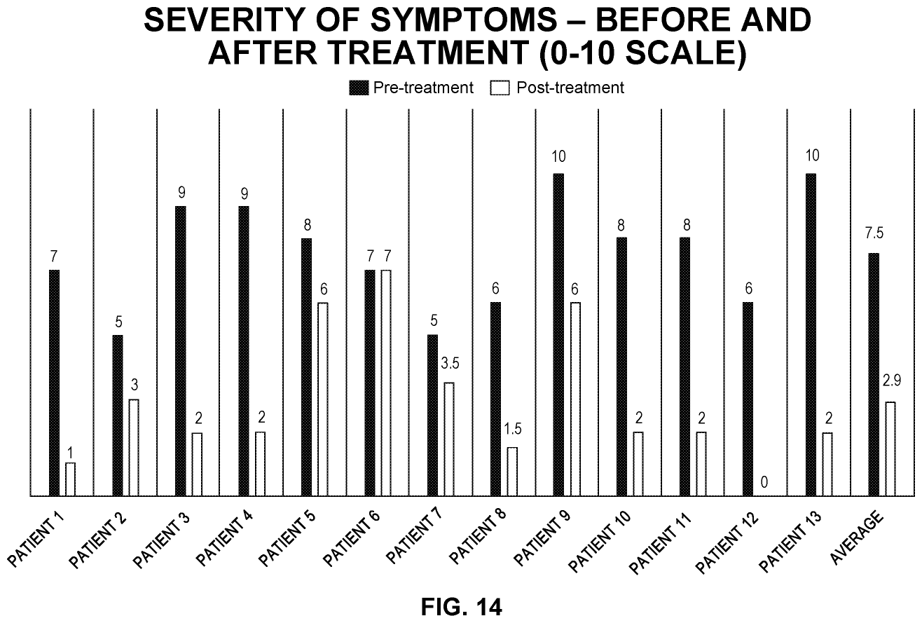

FIG. 14 shows treatment results for a plurality of users. FIG. 13 compares pre-treatment symptom(s) severity 1312 to post-treatment symptom(s) severity 1314. As shown in FIG. 13, for an individual user, symptom(s) severity can decrease by about 0 to about 8 points (on a 0-10) scale. Post-treatment symptom(s) severity 1314 can be less than about 75% of pre-treatment symptom(s) severity 1312. Specifically, post-treatment symptom(s) severity 1314 can be less than about 60% of pre-treatment symptom(s) severity 1312, less than about 50% of pre-treatment symptom(s) severity 1312, less than about 40% of pre-treatment symptom(s) severity 1312, or less than about 30% of pre-treatment symptom(s) severity 1312.

On average, post-treatment symptom(s) severity 1314 can decrease by about 4.5 points (on a 0-10 scale). An average post-treatment symptom(s) severity 1314 can be less than about 75% of an average pre-treatment symptom(s) severity 1312. Specifically, an average post-treatment symptom(s) severity 1314 can be less than about 60% of an average pre-treatment symptom(s) severity 1312, less than about 50% of an average pre-treatment symptom(s) severity 1312, or less than about 40% of an average pre-treatment symptom(s) severity 1312.

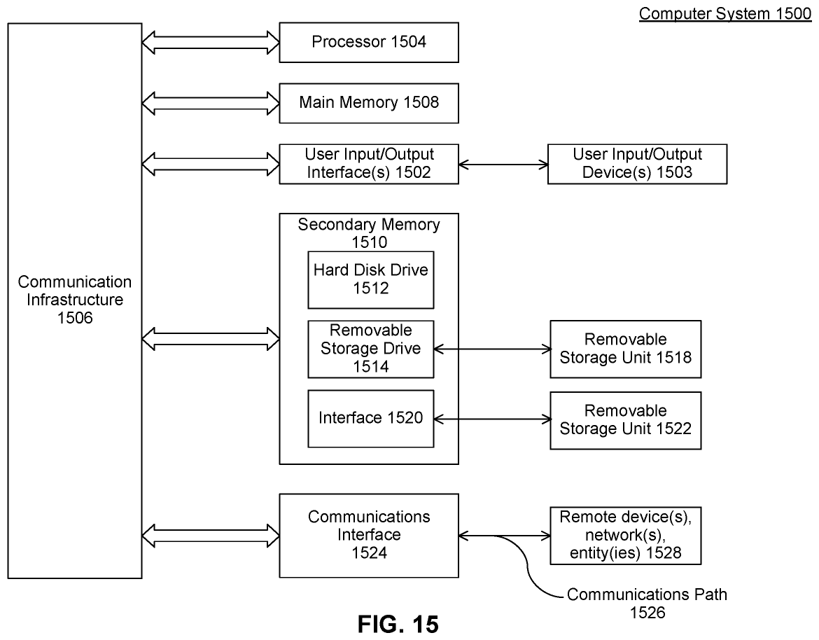

Various aspects can be implemented, for example, using one or more well-known computer systems, such as computer system 1500 shown in FIG. 15. One or more computer systems 1500 can be used, for example, to implement any of the aspects discussed herein, such as stimulator device 100, stimulator device 600, electronic device 700, server 1000, and/or method 1200, as well as combinations and sub-combinations thereof.

Computer system 1500 can include one or more processors (also called central processing units, or CPUs), such as a processor 1504. Processor 1504 can be connected to a communication infrastructure or bus 1506.

Computer system 1500 can also include user input/output device(s) 1503, such as monitors, keyboards, pointing devices, etc., which can communicate with communication infrastructure 1506 through user input/output interface(s) 1502.

One or more of processors 1504 can be a graphics processing unit (GPU). In an aspect, a GPU can be a processor that is a specialized electronic circuit designed to process mathematically intensive applications. The GPU can have a parallel structure that is efficient for parallel processing of large blocks of data, such as mathematically intensive data common to computer graphics applications, images, videos, etc.

Computer system 1500 can also include a main or primary memory 1508, such as random access memory (RAM). Main memory 1508 can include one or more levels of cache. Main memory 1508 can have stored therein control logic (i.e., computer software) and/or data.

Computer system 1500 can also include one or more secondary storage devices or memory 1510. Secondary memory 1510 can include, for example, a hard disk drive 1512 and/or a removable storage device or drive 1514. Removable storage drive 1514 can be a floppy disk drive, a magnetic tape drive, a compact disk drive, an optical storage device, tape backup device, and/or any other storage device/drive.

Removable storage drive 1514 can interact with a removable storage unit 1518. Removable storage unit 1518 can include a computer usable or readable storage device having stored thereon computer software (control logic) and/or data. Removable storage unit 1518 can be a floppy disk, magnetic tape, compact disk, DVD, optical storage disk, and/or any other computer data storage device. Removable storage drive 1514 can read from and/or write to removable storage unit 1518.

Secondary memory 1510 can include other means, devices, components, instrumentalities or other approaches for allowing computer programs and/or other instructions and/or data to be accessed by computer system 1500. Such means, devices, components, instrumentalities or other approaches can include, for example, a removable storage unit 1522 and an interface 1520. Examples of the removable storage unit 1522 and the interface 1520 can include a program cartridge and cartridge interface (such as that found in video game devices), a removable memory chip (such as an EPROM or PROM) and associated socket, a memory stick and USB port, a memory card and associated memory card slot, and/or any other removable storage unit and associated interface.

Computer system 1500 can also include a communications or network interface 1524. Communications interface 1524 can enable computer system 1500 to communicate and interact with any combination of external devices, external networks, external entities, etc. (individually and collectively referenced by reference number 1528). For example, communications interface 1524 can allow computer system 1500 to communicate with external or remote devices 1528 over communications path 1526, which can be wired and/or wireless (or a combination thereof), and which can include any combination of LANs, WANs, the Internet, etc. Control logic and/or data can be transmitted to and from computer system 1500 via communications path 1526.

Computer system 1500 can also be any of a personal digital assistant (PDA), desktop workstation, laptop or notebook computer, netbook, tablet, smart phone, smart watch or other wearable, appliance, part of the Internet-of-Things, and/or embedded system, to name a few non-limiting examples, or any combination thereof.

Computer system 1500 can be a client or server, accessing or hosting any applications and/or data through any delivery paradigm, including but not limited to remote or distributed cloud computing solutions; local or on-premises software (“on-premise” cloud-based solutions); “as a service” models (e.g., content as a service (CaaS), digital content as a service (DCaaS), software as a service (SaaS), managed software as a service (MSaaS), platform as a service (PaaS), desktop as a service (DaaS), framework as a service (FaaS), backend as a service (BaaS), mobile backend as a service (MBaaS), infrastructure as a service (IaaS), etc.); and/or a hybrid model including any combination of the foregoing examples or other services or delivery paradigms.

Any applicable data structures, file formats, and schemas in computer system 1500 can be derived from standards including but not limited to JavaScript Object Notation (JSON), Extensible Markup Language (XML), Yet Another Markup Language (YAML), Extensible Hypertext Markup Language (XHTML), Wireless Markup Language (WML), MessagePack, XML User Interface Language (XUL), or any other functionally similar representations alone or in combination. Alternatively, proprietary data structures, formats or schemas can be used, either exclusively or in combination with known or open standards.

In an aspect, a tangible, non-transitory apparatus or article of manufacture comprising a tangible, non-transitory computer useable or readable medium having control logic (software) stored thereon can also be referred to herein as a computer program product or program storage device. This includes, but is not limited to, computer system 1500, main memory 1508, secondary memory 1510, and removable storage units 1518 and 1522, as well as tangible articles of manufacture embodying any combination of the foregoing. Such control logic, when executed by one or more data processing devices (such as computer system 1500), can cause such data processing devices to operate as described herein.

Based on the teachings contained in this disclosure, it will be apparent to persons skilled in the relevant art(s) how to make and use aspects of this disclosure using data processing devices, computer systems and/or computer architectures other than that shown in FIG. 15. In particular, aspects can operate with software, hardware, and/or operating system implementations other than those described herein.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventors, and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The aspect(s) described, and references in the specification to “one aspect,” “an aspect,” “an example aspect,” “an exemplary aspect,” etc., indicate that the aspect(s) described can include a particular feature, structure, or characteristic, but every aspect may not necessarily include the particular feature, structure, or characteristic. Moreover, such phrases are not necessarily referring to the same aspect. Further, when a particular feature, structure, or characteristic is described in connection with an aspect, it is understood that it is within the knowledge of one skilled in the art to affect such feature, structure, or characteristic in connection with other aspects whether or not explicitly described.

Spatially relative terms, such as “beneath,” “below,” “lower,” “above,” “on,” “upper” and the like, can be used herein for ease of description to describe one element or feature's relationship to another element(s) or feature(s) as illustrated in the figures. The spatially relative terms are intended to encompass different orientations of the device in use or operation in addition to the orientation depicted in the figures. The apparatus can be otherwise oriented (rotated 90 degrees or at other orientations) and the spatially relative descriptors used herein can likewise be interpreted accordingly.

The term “about” or “substantially” or “approximately” as used herein means the value of a given quantity that can vary based on a particular technology. Based on the particular technology, the term “about” or “substantially” or “approximately” can indicate a value of a given quantity that varies within, for example, 0.1-10% of the value (e.g., ±0.1%, ±1%, ±2%, ±5%, or ±10% of the value).

Numerical values, including endpoints of ranges, can be expressed herein as approximations preceded by the term “about,” “substantially,” “approximately,” or the like. In such cases, other aspects include the particular numerical values. Regardless of whether a numerical value is expressed as an approximation, two aspects are included in this disclosure: one expressed as an approximation, and another not expressed as an approximation. It will be further understood that an endpoint of each range is significant both in relation to another endpoint, and independently of another endpoint.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

The claims in the instant application are different than those of the parent application or other related applications. The Applicant therefore rescinds any disclaimer of claim scope made in the parent application or any predecessor application in relation to the instant application. The Examiner is therefore advised that any such previous disclaimer and the cited references that it was made to avoid, may need to be revisited. Further, the Examiner is also reminded that any disclaimer made in the instant application should not be read into or against the parent application.

What is claimed is:

1. A device, comprising:
a stimulator for application to the tongue of a user, the stimulator configured to stimulate tongue muscle spindles via stochastic resonance, the stimulator comprising:
a transducer configured to produce vibrations,
a tongue pad configured to be coupled to the transducer,
a receiver configured to receive a signal comprising information about a type of the vibrations, the signal being generated using an application on an electronic device in wireless communication with the receiver,
an amplifier configured to amplify the signal, and
a controller configured to transmit the signal to the transducer to produce the type of the vibrations.

2. The device of claim 1, wherein the type of the vibrations is at least one of Brownian, pink, or white noise.

3. The device of claim 2, wherein the signal further comprises information about a frequency range of the vibrations.

4. The device of claim 3, wherein the frequency range is within about 1 Hz to about 300 Hz.

5. The device of claim 1, wherein the receiver is a personal area network (PAN) receiver.

6. The device of claim 1, wherein the transducer is an electromagnetic acoustic transducer.

7. The device of claim 1, wherein the transducer is a piezoelectric transducer.

8. The device of claim 1, wherein the tongue pad is configured to be in contact with a top surface of the tongue.

9. The device of claim 1, wherein the tongue pad is configured to be detachable from a body portion of the stimulator, and the tongue pad is one of a plurality of interchangeable tongue pads.

10. The device of claim 1, wherein the electronic device is a mobile phone.

11. A device, comprising:
a stimulator for application to the tongue of a user, the stimulator configured to stimulate tongue muscle spindles via stochastic resonance, the stimulator comprising:
a transducer configured to produce vibrations,
a tongue pad configured to be coupled to the transducer,
an amplifier configured to amplify a signal comprising information about a type of the vibrations, and
a controller configured to transmit the signal to the transducer to produce the type of the vibrations,
wherein the type of the vibrations comprises at least one of Brownian, pink, or white noise.

12. The device of claim 11, wherein the type of vibrations is selectable by a user between at least one of Brownian, pink, or white noise.

13. The device of claim 11, wherein the signal further comprises information about a frequency range of the vibrations.

14. The device of claim 13, wherein frequency range is within about 1 Hz to about 300 Hz and the type of the vibrations is Brownian noise.

15. The device of claim 11, wherein the transducer is an electromagnetic acoustic transducer.

16. The device of claim 11, wherein the transducer is a piezoelectric transducer.

17. The device of claim 11, wherein the tongue pad is configured to be in contact with a top surface of the tongue.

18. The device of claim 11, wherein the tongue pad is configured to be detachable from a body portion of the stimulator, and the tongue pad is one of a plurality of interchangeable tongue pads.

19. A method of treatment, comprising:
selecting a type of vibrations to be at least one of Brownian, pink, or white noise;
selecting a frequency range of the vibrations; and
applying a stimulator to the tongue of a user to apply the vibrations to the tongue of the user and stimulate tongue muscle spindles via stochastic resonance, the stimulator comprising:
a transducer configured to produce the vibrations, and
a tongue pad coupled to the transducer.

20. The method of treatment of claim 19, wherein the frequency range of the vibrations is within about 1 Hz to about 300 Hz.

21. The method of treatment of claim 19, wherein the frequency range of the vibrations is within about 20 Hz to about 250 Hz.

22. The method of treatment of claim 19, wherein the type of the vibrations is Brownian noise.

23. The method of treatment of claim 19, wherein the type of the vibrations is pink noise.

24. The method of treatment of claim 19, further comprising applying the stimulator to the tongue of the user for a time period within about 5 minutes to about 30 minutes.

25. The method of treatment of claim 24, wherein the time period is about 10 minutes to about 25 minutes.

26. The method of treatment of claim 19, wherein a post-treatment severity of a symptom of the user is less than about 75% of the pre-treatment severity of the symptom.

27. The method of treatment of claim 19, wherein a post-treatment severity of a symptom of the user is less than about 50% of the pre-treatment severity of the symptom.
